(19) United States
(12) Patent Application Publication
(10) Pub. No.: US 2025/0131994 A1
(43) Pub. Date: Apr. 24, 2025

(54) NON-FUNGIBLE TOKEN SYSTEM FOR ENSURING ETHICAL, EFFICIENT AND EFFECTIVE MANAGEMENT OF BIOSPECIMENS

(71) Applicant: University of Pittsburgh - Of the Commonwealth System of Higher Education, Pittsburgh, PA (US)

(72) Inventors: Marielle Sophia GROSS, Pittsburgh, PA (US); Balaji PALANISAMY, Pittsburgh, PA (US)

(73) Assignee: University of Pittsburgh - Of the Commonwealth System of Higher Education, Pittsburgh, PA (US)

(21) Appl. No.: 18/690,454

(22) PCT Filed: Sep. 14, 2022
(86) PCT No.: PCT/US2022/043481

§ 371 Date: Mar. 8, 2024

Related U.S. Application Data
(60) Provisional application No. 63/244,092, filed on Sep. 14, 2021.

(51) Int. Cl. G16H 10/60; G06F 21/62; G16H 80/00; H04L 9/32

(52) U.S. Cl. CPC G16H 10/60; G06F 21/6245; G16H 80/00; H04L 9/3268

(57) ABSTRACT

Embodiments described herein provide for a healthcare ecosystem of computing devices participating as nodes of a distributed ledger (e.g., blockchain). The distributed ledger includes NFTs representing corresponding patients, where the initial NET behaves logically as a token scaffold that represents a “digital twin” of the particular patient. The initial patient token represents personal profile data of patient personal data for the patient. The nodes may generate new data and/or new sub-tokens (or other blockchain entry type) representing new data, which the nodes associate with the token scaffold. The sub-tokens contain data for representing physical or digital specimens, health data, and data derived from the health data (e.g., cell lines, organoids, other model systems). As the nodes generate and associate new sub-tokens to the patient's token scaffold over time, the token scaffold graphs and facilitates integration of patient-specific data and evolves into a robust electronic health record (EHR) and digital twin.

### CROSS-REFERENCE TO RELATED APPLICATION

This application claims priority to U.S. Provisional Application No. 63/244,092, filed Sep. 14, 2021, which is incorporated by reference in its entirety.

### TECHNICAL FIELD

This application generally relates to systems and methods for managing distributed ledger technologies, such as blockchain implementations and non-fungible tokens (NFTs), in a healthcare ecosystem.

### BACKGROUND

The manner in which current healthcare systems generate, store, exchange, and manage various information gives rise to inefficiency, ineffectiveness, and injustice. Current research, treatment, and education from clinical care and research studies rely on stakeholder systems to disconnect patients from “secondary use” of patient-derived biospecimens (or “specimens”) and health data related to the patients or specimens. Research on human tissue and other patient-derived data produces medically relevant, personally significant and financially lucrative information and products. The technology, however, lacks mechanisms for fairly distributing benefits to patient-donors (primary stakeholders) or aligning patient interests with the benefits garnered by researchers and other “secondary” stakeholders.

Healthcare records are typically stored and persisted using conventional database technologies, such as cloud storage services that host a virtualized environment permitting healthcare stakeholders to store data pertaining the to the stakeholder's role. For example, a physician's office or hospital may store information in a database hosted by one cloud service, whereas clinical researchers at a university may store information in a database instance of a different cloud service licensed by the university. The stakeholders generate new data about a particular patient and store that data into the respective database systems, but the information remains siloed. The patients and other stakeholders (e.g., physicians) would greatly benefit from the ability to access different types of data generated or stored by the other stakeholders. While there are myriad business and personal benefits for such features, there are also business, statutory, and ethical issues associated with exchanging healthcare data between stakeholders. Overcoming these issues require technological solutions.

As an example, researchers regularly conduct research studies using biospecimens (or “specimens”) of patient-donors that the researchers receive from broker services or biobanks. While conducting the research study, the researchers often intentionally or incidentally produce information about the specimen and, by extension, about the patient's current or future health or likely healthcare needs, or those of the patient relatives (e.g. germline genomic data). The researchers, however, have no way of contacting the patient or the patient's healthcare provider, because the physician, broker service, or researcher typically disconnects the patient's personally identifiable information (PII) associated with the specimen prior to inclusion in research due to legal requirements, ethical standards, or institutional research protocols related to protection individual privacy.

What is needed is a means for providing researchers with custody of the specimens and the related specimen profile information required to conduct the research in a way that maintains patient privacy, while also maintaining provenance information associating the specimen with the patient-donor. In this way, the patient may benefit from the knowledge gained by the researchers and/or be compensated for the secondary use of the patient's specimen examined or used to create a secondary human cellular and/or tissue-based product during the research study, or reduce the volume of medical waste, as fewer biospecimens may remain untapped and unused as viable resources for research efforts.

### SUMMARY

Disclosed herein are systems and methods capable of addressing the above-described shortcomings and may provide any number of additional or alternative benefits and advantages. Embodiments described herein provide for a healthcare ecosystem of computing devices participating as nodes of a distributed ledger (e.g., blockchain). The distributed ledger includes NFTs representing corresponding patients, where the initial NFT behaves logically as a token scaffold that represents a “digital twin” of the particular patient. The initial patient token represents personal profile or patient personal data for the patient. The nodes may generate new data and/or new sub-tokens (or other form a blockchain entries) representing the new data, which the nodes associate with the token scaffold. The sub-tokens contain data for representing particular specimens (e.g., bio or in silica data, such as human cellular or tissue based product, saliva, radiology images, and the like), health data (e.g., genomic data), and data derived from the health data (e.g., cell lines, organoids). As the nodes generate and associate new sub-tokens to the patient's token scaffold over time, the token scaffold and sub-tokens represent a robust electronic health record (EHR) and digital twin of the patient. The token scaffolds and sub-tokens need not contain the various types of data, but may include the data representing each of the validated data sets and/or pointers to such data.

Stakeholders may access physical custody of specimens or various types of data for performing the particular functions of the particular stakeholder, by receiving data in the sub-tokens or otherwise available on the publicly available distributed ledger or blockchain. However, the information available to stakeholders cannot access the patient's PII or other private information due to encryption and obfuscation techniques applied to such data.

In an embodiment, a computer-implemented method for managing data for a blockchain comprises receiving, by a computer from a client device, patient personal data for a patient and a set of one or more validating inputs having validation data associated with one or more corresponding validating users; responsive to the computer validating the validation data of a threshold number of validation inputs from the set of one or more validation inputs, generating, by the computer, a patient token as a non-fungible token on a blockchain, the patient token representing the patient personal data and including a unique patient token identifier; receiving, by the computer from the client device, specimen data for a specimen associated with the patient, the specimen data including a unique specimen identifier based at least in part on the patient personal data; and generating, by the computer, a specimen sub-token of the blockchain representing the specimen data using at least a portion of the patient personal data of the patient token, the specimen sub-token including the unique specimen identifier, a unique specimen token identifier, the unique patient token identifier, and an encrypted version of the portion of the patient personal data.

In another embodiment, a system comprises a computer comprising a processor configured to: receive, from a client device, patient personal data for a patient and a set of one or more validating inputs having validation data associated with one or more corresponding validating users; responsive to validating the validation data of a threshold number of validation inputs from the set of one or more validation inputs, generate a patient token as a non-fungible token on a blockchain, the patient token representing the patient personal data and including a unique patient token identifier; receive, from the client device, specimen data for a specimen associated with the patient, the specimen data including a unique specimen identifier based at least in part on the patient personal data; and generate a specimen sub-token of the blockchain representing the specimen data using at least a portion of the patient personal data of the patient token, the specimen sub-token including the unique specimen identifier, a unique specimen token identifier, the unique patient token identifier, and an encrypted version of the portion of the patient personal data.

In another embodiment, a computer-implemented method for managing data access for blockchain data comprises obtaining, by the computer, a specimen request associated with a researcher device, the specimen request including one or more specimen attributes; determining, by the computer, a cohort of one or more specimens having specimen data satisfying the one or more specimen attributes by querying the specimen data of a plurality of specimen sub-tokens of one or more blockchains using the one or more specimen attributes of the specimen request; generating, by the computer, one or more custody updates to one or more transaction blocks on a blockchain having the patient token, the one or more custody updates corresponding to the one or more specimens of the cohort, wherein the researcher device does not have access to patient personal data and a unique patient identifier; receiving, by a computer, from the researcher device research results data for a specimen of the cohort, the research results data including a unique specimen identifier for the specimen; and generating, by the computer, a research results sub-token representing the research results data using at least a portion of the research results data and a specimen unique identifier associated with a patient unique identifier of a patient token.

In another embodiment, a system comprises a computer configured to: obtain a specimen request associated with a researcher device, the specimen request including one or more specimen attributes; determine a cohort of one or more specimens having specimen data satisfying the one or more specimen attributes by querying the specimen data of a plurality of specimen sub-tokens of one or more blockchains using the one or more specimen attributes of the specimen request; generate one or more custody updates to one or more transaction blocks on a blockchain having the patient token, the one or more custody updates corresponding to the one or more specimens of the cohort, wherein the researcher device does not have access to patient personal data and a unique patient identifier; receive from the researcher device research results data for a specimen of the cohort, the research results data including a unique specimen identifier for the specimen; and generate a research results sub-token representing the research results data using at least a portion of the research results data and a specimen unique identifier associated with a patient unique identifier of a patient token.

In another embodiment, a computer-implemented method comprises receiving, by a computer, research data or treatment data for a patient represented by a patient token of a blockchain, a physician identifier, and a set of one or more validating inputs purportedly validating the research data or treatment data from a physician device; generating, by the computer, a treatment sub-token representing the research data or treatment data using a physician unique identifier and at least a portion of patient data for the patient, responsive to the computer validating the validation data of a threshold number of validation inputs from the set of one or more validation inputs; receiving, by the computer, from a patient device a claim instruction and a patient validating input; and in response to the computer validating patient validation data of the patient validating input: updating, by the computer, one or more transaction blocks of the blockchain to indicate an association between the patient token or the treatment sub-token and one or more identifiers associated with the patient. For example, a patient with a triple negative breast cancer sample represented by an NFT scaffold and one or more sub-tokens may receive certain chemotherapy treatment, followed by surgery, where each treatment (e.g., chemotherapy, surgery) would be recorded in the NFT scaffold and sub-tokens as health transactions of the digital twin.

In another embodiment, a system comprises a computer comprising a processor configured to: receive research data or treatment data for a patient represented by a patient token of a blockchain, a physician identifier, and a set of one or more validating inputs purportedly validating the research data or treatment data from a physician device; generate a treatment sub-token representing the research data or treatment data using a physician unique identifier and at least a portion of patient data for the patient, responsive to the computer validating the validation data of a threshold number of validation inputs from the set of one or more validation inputs; receive from a patient device a claim instruction and a patient validating input; and in response to the computer validating patient validation data of the patient validating input: update one or more transaction blocks of the blockchain to indicate an association between the patient token or the treatment sub-token and one or more identifiers associated with the patient.

In another embodiment, a computer-implemented for managing data for a blockchain comprises receiving, by a computer from a client device, physician data for a healthcare provider and a set of one or more validating inputs having validation data associated with one or more corresponding validating users; responsive to the computer validating the validation data of a threshold number of validation inputs from the set of one or more validation inputs, generating, by the computer, a physician token as a non-fungible token on a blockchain, the physician token representing the physician and including physician profile information of the physician data, a unique physician identifier, and a unique physician token identifier; and generating, by the computer, one or more physician-related sub-tokens of the blockchain representing at least a portion of the physician data, each physician-related sub-token including a unique sub-token identifier, the unique physician token identifier, and an encrypted version of the unique physician identifier.

In another embodiment, a system comprises a computer comprising a processor configured to: receive from a client device physician data for a healthcare provider and a set of one or more validating inputs having validation data associated with one or more corresponding validating users; responsive to the computer validating the validation data of a threshold number of validation inputs from the set of one or more validation inputs, generate a physician token as a non-fungible token on a blockchain, the physician token representing the physician and including physician profile information of the physician data, a unique physician identifier, and a unique physician token identifier; and generate one or more physician-related sub-tokens of the blockchain representing at least a portion of the physician data, each physician sub-token including a unique sub-token identifier, the unique physician token identifier, and an encrypted version of the unique physician identifier.

In another embodiment, a computer-implemented for managing data for a blockchain comprises receiving, by a computer from a client device, researcher data for a researcher and a set of one or more validating inputs having validation data associated with one or more corresponding validating users; responsive to the computer validating the validation data of a threshold number of validation inputs from the set of one or more validation inputs, generating, by the computer, a researcher token as a non-fungible token on a blockchain, the researcher token representing the researcher and including researcher profile information of the researcher data, a unique researcher identifier, and a unique researcher token identifier; generating, by the computer, one or more researcher sub-tokens of the blockchain representing at least a portion of the researcher data, each researcher sub-token including a unique sub-token identifier, the unique researcher token identifier, and an encrypted version of the unique researcher identifier.

In another embodiment, a system comprises a computer comprising a processor and configured to: receive from a client device researcher data for a researcher and a set of one or more validating inputs having validation data associated with one or more corresponding validating users; responsive to the computer validating the validation data of a threshold number of validation inputs from the set of one or more validation inputs, generate a researcher token as a non-fungible token on a blockchain, the researcher token representing the researcher and including researcher profile information of the researcher data, a unique researcher identifier, and a unique researcher token identifier; and generate one or more researcher sub-tokens of the blockchain representing at least a portion of the researcher data, each researcher sub-token including a unique sub-token identifier, the unique researcher token identifier, and an encrypted version of the unique researcher identifier.

It is to be understood that both the foregoing general description and the following detailed description are exemplary and explanatory and are intended to provide further explanation of the invention as claimed.

### BRIEF DESCRIPTION OF THE DRAWINGS

The present disclosure can be better understood by referring to the following figures. The components in the figures are not necessarily to scale, emphasis instead being placed upon illustrating the principles of the disclosure. In the figures, reference numerals designate corresponding parts throughout the different views.

FIG. 1A shows data flow for a blockchain implementation of a distributed ledger of a healthcare computing ecosystem, according to an embodiment.

FIG. 1B shows components of a system implementing healthcare NFT-based token scaffolds, according to an embodiment.

FIG. 2 shows execution steps of a method for initializing a new token scaffold for a new patient, according to an embodiment.

FIG. 3 shows execution steps of a method for third-party access to a portion of a token scaffold and updates to the token scaffold, according to an embodiment.

FIG. 4 shows execution steps of a method for physician access to the token scaffold and updates to the token scaffold, according to an embodiment.

FIG. 5 depicts an example of data flow of components of a system implementing healthcare NFT-base token scaffolds, according to an embodiment.

### DETAILED DESCRIPTION

Reference will now be made to the illustrative embodiments illustrated in the drawings, and specific language will be used here to describe the same. It will nevertheless be understood that no limitation of the scope of the invention is thereby intended. Alterations and further modifications of the inventive features illustrated here, and additional applications of the principles of the inventions as illustrated here, which would occur to a person skilled in the relevant art and having possession of this disclosure, are to be considered within the scope of the invention.

### Overview of Certain Disclosed Features

Described herein are systems and methods for securely storing, distributing, and tracking health-related information about people using one or more aspects of distributed ledger technology, such as blockchains or non-fungible tokens (NFTs), sometimes referred to as “tokens.” Embodiments include a system for a secure and ethical information marketplace, such as patient information, specimen information, research information, dynamic informed consent, and treatment information, among others. The system includes any number of computing devices participating as nodes of the distributed ledger. An expandable set of tokens and sub-tokens, referred to as a “token scaffold,” of the distributed ledger represent each particular person (e.g., patient). The system uses the token scaffold to store and track the various types of information, transactions, and custody responsibilities of specimens on behalf of various actors (e.g., patients, physicians, biobanks, researchers, healthcare commerce entities) participating in the distributed ledger ecosystem. The token scaffold includes an initial token (sometimes referred to as a “patient token”) for the particular patient and any number of sub-blocks (sometimes referred to as “sub-tokens”) for particular types of data related to the patient. The sub-blocks contain data related to or representing, for example, individual specimens, health data, and derivative products (e.g., cell lines, organoids), among other types of data. Over time, the token scaffold's components (e.g., patient token, sub-blocks) ultimately represent a digital twin of the patient that evolves, grows, and represents the patient with an immutable record of the patient's health care and related transactions.

Embodiments include nodes that participate and implement a public or private blockchain, or other form of distributed ledger, containing blockchain entries, such as blocks, tokens (e.g., NFTs). Embodiments may include and implement any number of blockchain technologies (e.g., NFTs, distributed applications (DAPPs), smart contracts), or blockchain platforms (e.g., Ethereum, Polygon, Solana, Tezos, Tron) and related standards, such as Ethereum standards for NFTs (e.g., ERC-20, ERC-721) and multi-tokens (ERC-1155, TZIP-012, TRC-721), among others. The terms “blockchain,” “blocks,” and “tokens” are used herein merely to describe certain aspects of example embodiments and are not necessarily limiting on technologies implemented in potential embodiments.

Similarly, the terms “sub-blocks” and “sub-tokens” may refer to hierarchical, logical relationships between entries on the blockchain, but also should not be considered limiting on the potential technologies. As an example, a node of the blockchain may mint a new patient NFT representing an new patient on the blockchain, while the same or different node may mint a new biosample NFT or (other type of blockchain entry) representing a biosample taken from the new patient. In this example, the new biosample NFT may be considered a sub-block or sub-token (hierarchical, logical child) of the new patient NFT (hierarchical, logical parent). In some embodiments, the blockchain standards provide for sub-tokens or sub-blocks. But blockchain standards need not implement sub-tokens or sub-blocks, as such. For example, Ethereum standards (e.g., ERC-1155, ERC-721) do not provide for hierarchical “sub-tokens” or “sub-blocks.” However, the embodiments that implement Ethereum may establish notional, hierarchical sub-block or sub-token relationships using some linkage between “parent” blocks and “child” blocks. For instance, when implementing Ethereum (e.g., ERC-1155, ERC-721), a node may create a parent token (e.g., patient NFT) and a sub-token (e.g., biosample NFT) containing linking identifiers (or other linking data) between the parent token identifier (e.g., patient NFT identifier) and the sub-token identifier (e.g., biosample NFT identifier). The blockchain includes, for example, a patient token containing details about the patient, and any number of biosample sub-tokens containing the details about the biosamples collected from the patient. Linking data, such as token identifiers or patient identifiers, represent a link between the core patient token (as a hierarchical parent token) and the related biosample tokens (as a hierarchical child token). In this way, the patient token represents, and is sometimes referred to as, a “patient scaffold” associated with each sub-token relevant to the patient.

Moreover, the terms “tokens,” “blocks,” “sub-tokens,” and “sub-blocks” refer to examples of entries of the blockchain and do not limit potential embodiments. For example, a patient token representing a patient may be a typical blockchain block or an NFT. As another example, a biosample sub-block representing a biosample specimen may be a block or an NFT. Accordingly, for ease of description, the terms “tokens” and “blocks” may be used interchangeably in reference to entries of the blockchain. Likewise, for ease of description, the terms “sub-tokens,” and “sub-blocks” may be used interchangeably in reference to entries of the blockchain that are hierarchical, logical children to one or more parent entries on the blockchain.

Each token or sub-token includes a particular type of data or a pointer (e.g., hyperlink) to a non-transitory storage medium containing some or all of the particular type of data. A participating node generates the initial token representing patient personal data for the patient. Non-limiting examples of the patient personal data for the patient profile data may include: a patient name; date, time and location of birth; biometric data and identifiers (e.g., fingerprints, photographs); unique genetic data (e.g., some or all of the genome sequence); omic data (e.g., proteome, microbiome, social mediome); identity of parents, including biological, birth parents and guardians; health status (e.g., living, deceased, breast cancer, hypertension, diabetes, allergies); proxies or other representatives; physician identities; designated or elected stewards of data or property and nominees information; locations associated with the patient (e.g., home address(es), city/state); patient identifiers (e.g., state ID, national ID, international ID, SSN, driver's license, passport number); medical record identifiers from each health system/provider where they receive care and other customer identification numbers (e.g., assigned by direct to consumer genetics or ancestry companies, clinical trial platforms, health apps); and current specimen sub-block inventory, among others.

Each person is associated with and assigned only a single NFT scaffold (e.g., one patient per NFT scaffold). An NFT scaffold is in a one-to-one ratio with a patient, whereas the sub-tokens will be essentially limitless, sometimes representing finite solid-state assets, reproducible assets, or digital assets. The sub-tokens may correspond to different properties or aspects of the individual person/patient represented by the NFT. In some implementations, however, a person may be associated with an assigned additional or alternative NFT scaffold related to a particular role (e.g., researcher, physician), such as a researcher NFT scaffold or physician NFT scaffold. The term “physician” is not intended to be limited to a Medical Doctor (MD), but may refer to users having the role of a type of healthcare provider, such as a psychologist, nurse, nurse practitioner, physician assistant, or pharmacist, among others. As used herein, the term “physician” generally refers to users or groups of users (e.g., medical practice, hospital team, healthcare clinic) having these or similar types of healthcare provider roles. In some cases, the healthcare provider or “physician” may include an artificial intelligence computing service or program that performs diagnostic or treatment counseling related to a patient's healthcare. Similarly, a “researcher” is not necessarily limited to users, groups of users, or entities conducting research on the physical and/or digital biospecimens, but may include any third-party users (or groups of users) having roles accessing the biopecimens or related biospecimen data, as compared to a “physician” (and other healthcare providers) whose role includes a direct or close relationship with the patient. These role-based scaffolds function as a “skin” or “avatar” that generate sub-tokens based on that particular role, and may unlock certain features for the particular user when the user accesses a software application using the selected role (e.g., physician role or patient role). The application can have different user-interface environments (e.g., set of features and functions), which are customized to the particular roles. The roles may also have relative controlled access to patients' information in the corresponding NFT scaffolds. For example, physicians may have broader access rights to patient information than researchers, though physicians may be prohibited from updating research information generated by the researchers.

Additional nodes generate sub-blocks using the distributed ledger technology. The nodes generate the sub-blocks based upon certain identifier values of the initial token or an earlier sub-block. The sub-block represents various types of data related to the patient, such as specimen data, treatment data, and research result data (e.g., participation and relevant research information), among others. The nodes append the sub-blocks to the token scaffold, such that, over time, the tokens and sub-blocks of the token scaffold represent a digital twin with an immutable health record for the particular patient that is system agnostic.

A system or ecosystem comprises any number of computing devices participating as nodes to the distributed ledger, or otherwise contributing to the operations described herein. A computing device (e.g., server) generates the token scaffold for the patient according to one or more distributed ledger technologies (e.g., blockchain, NFTs, smart contracts). The token scaffold includes the patient token representing the patient personal information about the patient. In addition, the patient token includes one or more identifiers, including uniquely identifying patient personal information for the particular patient. For instance, the initial block represents the patient personal information (e.g., social security number (SSN), date of birth (DOB), name) and a global unique identifier (GUID) for the patient. The server or other computing devices participating as nodes of the distributed ledger may generate and append additional sub-blocks representing various types of data to the patient's token scaffold.

As an example, the server may generate the initial token and the token scaffold in conjunction with issuing a birth certificate when the patient is born, though embodiments may generate the initial block and the token scaffold at any time. A user (e.g., the person, physician, healthcare worker, government administrator, social worker, researcher) may generate the initial block at any arbitrary time using a computing device having access to the system over the Internet (e.g., as an embryo, fetus, an infant, a child, an adult, posthumously). The healthcare workers may collect various specimens (e.g., placenta, umbilical cord, mother's blood, child's blood) while treating the mother through labor and following the birth. The server or other device may generate specimen sub-blocks representing specimen data for each respective specimen, which the server associates with the token scaffold of the patient (i.e., the newborn). The server may also associate all or some of the specimen sub-blocks with the mother's token scaffold, and vice versa. For example, if at the time of birth the scaffold is created for a newborn, a scaffold may be created for the mother if the mother does not yet have an associated NFT scaffold digital twin.

Embodiments may include one or more certificate authorities that issue, manage, evaluate, and revoke cryptographic digital certificates and encryption keys (e.g., asymmetric public-private key pairs, symmetric keys). The certificate authorities may, for example, certify new data for new NFTs (e.g., patient token, sub-blocks) or verify a purported patient-user attempting to associate (or “claim”) a new NFT with the patient's scaffold. The NFTs includes certificate information or unique identifiers as issued by the certificate authorities or derived from such unique identifiers. At any point of time, the certificate information contained in the NFT verifies the authenticity and validity of the NFT. Periodic revalidation will be required on a regular basis. For example, when a patient corresponding to a particular token scaffold expires, the certificate authority indicates the patient certificate and patient identifier expired, which propagates to each certificate or identifier of the sub-blocks in the patient scaffold. Though the patient is no longer alive, the patient's token scaffold persists in the distributed ledger as a digital twin for the patient. The server or other node updates the patient personal information in a database or in a transaction entry on the blockchain to indicate the patient's death and indicate that the ownership rights for the scaffold assets transferred to one or more proxy users. If appropriate and desired, the server or other node generates new sub-blocks for the patient's organs or cadaver. Of note, permanent association with relatives of the patient who expired will remain active, such that research or other use of the expired patient sub-tokens will be traceable and providing feedback to the digital twins of relatives for whom learning about the patient who expired may be relevant to the health, personal, or financial wellbeing of the surviving individuals.

NFT scaffolds or sub-tokens may also be created directly by biobanks or research laboratories, for example, for each of the corresponding patient identifiers, even if the biobank user or device does not know the identity of the corresponding patient. Generally, biobanks manage biorepositories that track, manage, store, and distribute any number of physical and/or digital biospecimens or biosamples (e.g., organs, organoids). The devices of biobanks (e.g., servers, databases, client devices) execute software that, for example, tracks custody or access privileges to biosamples, processes requests for access rights or custody transfers for biosamples, stores biosample data (sometimes referred to as “biospecimen data” or “specimen data”) related to physical or digital biosamples, and updates access rights or custody information as biosamples are distributed or transferred, to or from the biobank. In some cases, the NFT may be a placeholder or “shadow” NFT, which sub-tokens generated by the biobank's users may associate new sub-tokens. Although the identity of the patient is unknown, the sub-token for a specimen may contain any number of potential characterizations, including where and when the specimen was obtained, what research has been involved with the specimen, and any products or information derived using the specimen (e.g., biologics, publications, patents). Meanwhile the same patient may have claimed the NFT scaffold and/or sub-tokens, thereby adding the biobank-generated NFT and/or sub-token to the patient's NFT scaffold.

A local trusted entity with administrative controls over the distributed ledger and/or certificate authority may record the research outcomes onto corresponding sub-tokens of NFT scaffolds involved in a research study. Devices of such central entity may execute smart contract programming instructions or other programmable mechanisms to perform these operations autonomously, based on pre-defined, revisable terms reflective of the status and nature of the given research.

The validity and authenticity of the sub-blocks are verified using certificate authorities to monitor and track custody of specimens in a supply chain for specimens represented by corresponding specimen sub-blocks. For example, a specimen sub-block for a tissue sample may validated by the newborn's mother, the physician, the midwife, the nurse, and/or a biobank administrator who oversees banking of excess tissue, among other types of users. The certificate authority generates the certificates public key cryptography, and in some cases using digital signatures of the particular user receiving the certificate.

The distributed ledger includes any number of smart contracts driving behavior of the nodes (e.g., centralized server, client computing device) of the ecosystem. The smart contracts instruct the nodes to perform certain operations associated with the various transactions or features described herein. The node executes the programming instructions of the smart contract based upon a particular transaction and relative to the role of the users involved with the particular transaction (e.g., generate new sub-block, update custody transaction data, validate user certificates, request or revoke patient consent).

The patient scaffold can ensure the patient remains a beneficiary of the downstream clinical and non-clinical (e.g., research results) testing performed on the patient's specimens, as represented by the updating sub-tokens. NFTs help maintain structural integration of care and research by enabling the nodes to reference or trace the patient's unique identifier (e.g., cryptographic certificate, arbitrary value based on the cryptographic certificate) through current and future specimen uses that may occur inside or outside of existing clinical pathways. A server or other device generates a specimen sub-block based, in part, upon the patient's uniquely identifying information, which irrevocably binds the specimen to the patient when the specimen is used in research and therefore the patient is a research subject. Even though the researcher does not have access to the patient's identifying information and cannot know or identify the patient-donor, the patient can remain informed about the results of the clinical testing involving the patient's specimen. Another benefit of implementing such features includes improved translation of existing knowledge regarding genomic/genetic risk factors for certain health concerns for patients and relatives into personalized screening and management strategies. This may be especially critical for enabling just-in-time translation of the outcomes of experimentation on “living” biobank digital specimens (e.g., organoids, PDX models, and related blends, like organoids-on-a-chip) directly into the care of the source patient.

A researcher may access or generate specimen sub-blocks, and update aspects of the patient scaffold or other scaffolds (e.g., a researcher scaffold from a researcher token). This beneficially enables a decentralized exchange of biospecimen sub-tokens as tokenized biosamples representing the biospecimen (sometimes referred to as a “biosamples” or “samples”). Moreover, such features may automate operations for optimal distribution of access to the biospecimen, and/or may automate operations of returning value back to the patients whose samples are used by the researcher (value may be in the form of health, personal/social, or financial benefits). Furthermore, when biobanks and researchers participate in the features and functions discussed herein, biobanks serving as the source of the samples provide the biospecimen sub-tokens to researchers in a way that makes the underlying samples accessible to the researchers who would not otherwise have access to those samples stored with the biobank.

Embodiments include mechanisms for validating the preexisting data (e.g., provenance, data, tissue stewardship systems), each of which rely on local trusted entities, document them on blockchain and provide subsequent validations of the internal and external consistency of the provenance identities, enabling legacy system entries to become more robust, utilizing data correlation methods and ultimately enhance trustworthiness and interoperability, with decreased reliance on local trust points over time via subsequent validations. In some cases, for example, an individual biobank may generate donor or consent tokens (or donor or consent sub-tokens) to represent donor patients by incorporating a series of previously recorded data signatures and timestamps from the electronic medical record stored in a database or in a blockchain entry, each of which may be related to specific transactions between validated users in the existing system. Such a token may incorporate validating and identifying details of a patient personal data or obfuscated patient information, such as the first two letters of the first and last name of the patient donor, the individual (e.g., patient, donor, parent/guardian) who consented to the biobank donation of an associated specimen, and identifying information about the individual who scanned the paper consent into the electronic file system, respectively, along with the written date/time for the first two as well as the date/timestamp of the scanned data entry. A consent token or consent sub-token may further include a consent indicator as described herein.

In some embodiments, a centralized control system integrate or properly locate given sub-tokens that have been previously separated from the respective original patient original NFT scaffold. In some cases, an “owner” (custodian) of a biospecimen or similar type of biosample, such as a biobank or researcher, may create “shadow” NFTs that represent an unknown patient with whom the custodian may associate one or more new sub-tokens. When the respective patient or proxy representative claims the patient's NFT scaffold, and the scaffold begins to be populated by sub-tokens, the system will periodically “check” the shadow NFT scaffolds to assess if such shadow NFT scaffolds (e.g., tokens and/or sub-tokens) related to the patient and should be associated with the patient's NFT scaffold. For example, the system may identify research cohorts (groups of people with similar features) and determine if the people in an identified research cohort are, in fact, the same person. The system enables the patient's NFT scaffold to absorb the related shadow NFTs if the similarities in the features satisfy a similarity threshold. The patient's NFT scaffold may integrate one or more shadow NFT scaffolds representing currently disconnected sub-token collections for the same patient via a machine-executed process that verifies and executes the integration of the shadow NFT scaffold and/or shadow sub-tokens having similar data of the patient's NFT scaffold and/or patient's sub-tokens.

The software application of the system may allow users of the various roles to access and manipulate the various types of data and sub-tokens, and perform various operations and blockchain transactions. For example, patients have access to the scaffold to oversee and engage with a proposed research project that requested the patient's NFT scaffold. As another example, patients may seek out and connect with other patients in a cohort of patient-users having common features (e.g., background information, treatment, disease), allowing the patients in the cohort to leverage similar sub-tokens as a collective. The patients' anonymity is preserved as the patients in the cohort would not have access to any identifying information (e.g., each patient's identity, location, name, and other information would remain obfuscated) except as permitted or revealed by the patient. Beneficially, these features and functions enable devices to automate and integrate the implementation of precision medicine based on facilitating the aggregation and holistic appraisal of what was learned by each separate actor-user who interacted with or tracked patient biospecimens The patient can agree via the software application inputs to grant the cohort of patients or sub-cohorts to see certain aspects of the patient's NFT or sub-tokens. For instance, the patient may grant the cohort, sub-cohort, or specific patients access to a particular sub-token or data (e.g., name, photo (of the patient or an image they generate/select/avatar to represent them), city, state).

Certain aspects described herein may be implemented without a blockchain related or NFT platform. Although such a solution may provide different security properties, the underlying benefits of tracking, privacy, and oversight remain available. Embodiments may include a system employing a centralized approach to certain features without blockchains and NFTs. For example, a server (or a set of servers) and a centralized database (e.g., relational database) at a service platform can host data related to, for example, which entity or user has possession (or ownership) over certain patient biosamples, and/or maintain information about which research studies make use of which biosamples. A centralized database and application may track and query research results produced by the research studies, and return the query and research study results back to the appropriate users (e.g., patients, physicians). Oftentimes, however, such a centralized approach provides different kind of security protection. For instance, if there was an attack on the centralized server and if an attacker is able to modify the ownership information on the servers, then the integrity of the ownership information will be lost. A blockchain NFT-based approach on the other hand incorporates protection through decentralized consensus protocols of the underlying blockchain, as executed by one or more node computers participating in the blockchain according to embodiments herein. In general, the integrity of the information stored in the blockchain cannot be compromised unless a majority of the nodes in the network collude to violate the integrity. A centralized server-based implementation, as implemented in certain embodiments described herein, may beneficially offer additional, alternative, or various different types of security protection compared to typical blockchain NFTs that are decentralized. In this way, features and functions herein (e.g., tracking and retaining ownership of patient information, connecting researchers with patients and/or patient samples (e.g., tissues, organoids, biosamples), returning results to the patients) are securely accessible to and executed by nodes of the blockchain or computing devices associated with a healthcare data system (e.g., authority servers, biobank servers, client devices). As such, the features and functions benefit from security, efficiency, and centralized control, among other benefits, afforded by decentralized and centralized approaches.

While on-chain delivery of research results is a feature of certain embodiments, in some embodiments, research results may be delivered to patients (or other types of users) privately, off-chain. While all of the biospecimen data will be referenced on-chain, the actual biospecimen data itself can be maintained off-chain. One or more databases may store the various types of user information and biosample data in a backend non-transitory storage medium, on the backend, off-chain. In such embodiments, the biospecimen data included in the biospecimen token includes pointers or other references to the stored off-chain biosample data. The database is accessed by a node accessing the biospecimen information (or other types of information stored off-chain) when presenting the off-chain data on the frontend of the application. The database serves as a storage of data off-chain accessible within the service application and maintained privately by the centralized authority hosting the service application. The database stores all the data about, for example, the patient profile, researchers, and the biosamples in the system. While the database also stores data indicating the ownership or possession of the biosamples, the same ownership information is captured using the NFTs on the blockchain. The NFTs on the public blockchain are immutable. The off-chain database storage helps manage private information about the patients and the patient biosamples within the database of the service application, without making the private patient information or biospecimen information available on the public blockchain.

The scaffold of tokens represents an expansive conceptualization of the individual human and also the varieties of related data produced from/about/by users before, during, and after a lifetime, along with the series of non-fungible relationships that connect this individual to others. There will be instances in which there is an advantage or critical need for the patient, or physician/researcher, to sequester their identities within the NFT scaffold, and thus giving rise to a potential need for superimposable identities within the NFT scaffold framework. For example, when privacy interests are in tension with the interests of having a holistic picture of the patient, it may be in a patient's best interest to disaggregate the components of their NFT scaffold relative to their reproductive health (e.g., undergoing an abortion, evaluation or treatment for sexually transmitted diseases, etc), or to mental health, or other stigmatized conditions, wherein the benefits of keeping these histories sequestered may significantly outweigh the benefits of including such information in a central patient dashboard/identity. To this extent, a patient's scaffold may be delineated either as a distinct scaffold, or as sub-scaffold, that can be integrated when aggregation is desired or disaggregated when desired. Additionally, these various types of user scaffolds may be representative of an individual's relationship with a particular institution, set of norms, qualifications, or standards, which may be in conflict with users' various activities in certain contexts. The ability to disaggregate or otherwise disentangle these activities and identities may therefore be useful in facilitating the management of and minimizing conflicts of interest. For example, a physician may have certain identities, data and relationships related to their provision of clinical care in one setting compared to engagement in scholarly or commercial activity in other social or business contexts, or even, potentially, in providing clinical care in other contexts/system architectures.

### Example System Components

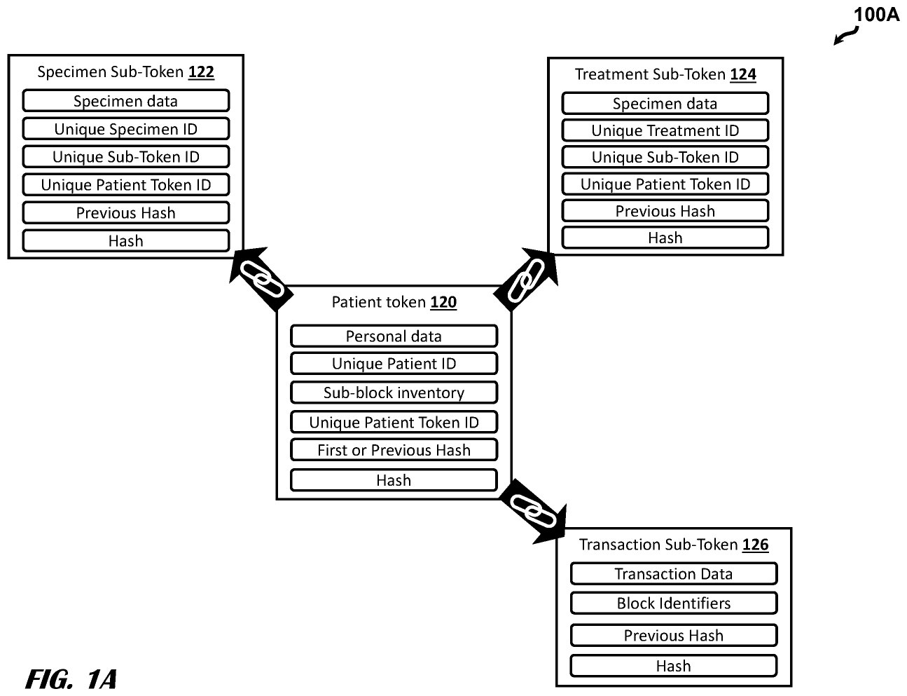

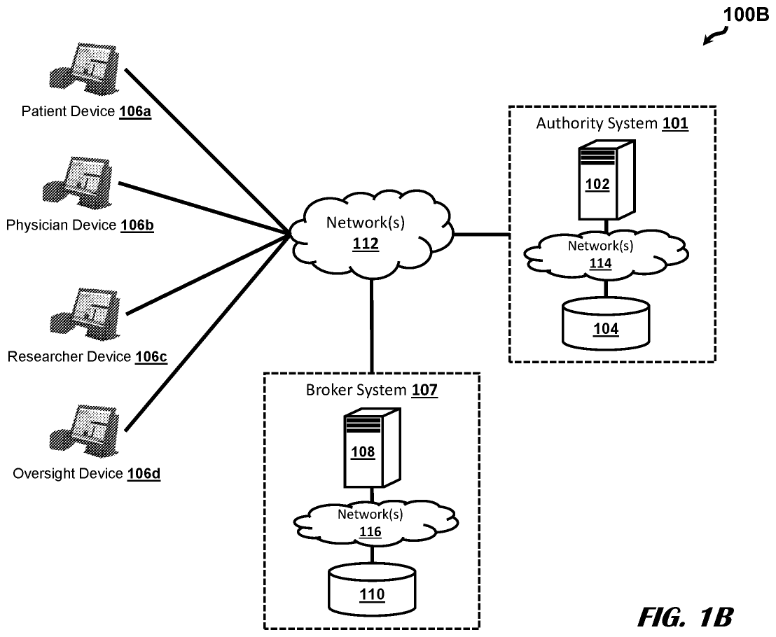

FIGS. 1A and 1B illustrate a blockchain 100a (or other form of distributed ledger) and a system 100b of networked computing devices, collectively referred to as a healthcare computing ecosystem 100.

FIG. 1A shows data flow for the blockchain 100a implemented by computing devices participating as nodes of the blockchain 100a. The blockchain 100a may include any number of blocks representing various types of data. The nodes may generate the blocks according to typical processes for generating interrelated blockchain blocks and/or using processes for generating NFTs. For instance, the blockchain 100a comprises a patient token 120, specimen sub-token 122, and treatment sub-token 124. An authority server 102 (or other node of the system 100a) generates the patient token 120 as an NFT representing personal information data for a particular patient, and generates the specimen sub-token 122 representing specimen data and the treatment sub-token 124 representing treatment data as sub-blocks. The patient token 120 serves as the first block of a collection of blocks associated with the patient, referred to as token scaffold (also referred to as a “patient scaffold” or “NFT scaffold”), forming a digital twin representation of the patient.

The ecosystem 100 implements the blockchain 100a as a distributed ledger of the ecosystem 100, though embodiments may implement any form of distributed ledger, such as a ledger table or the blockchain 100a. Embodiments may also implement a combination of distributed ledger forms. For example, the blockchain 100a includes transaction sub-tokens 126 that nodes (e.g., authority server 102, broker server 108) generate to represent various types of on-chain or off-chain transactions. The nodes generate these transaction sub-tokens 126 on a portion of the blockchain 100a parallel from (or in-line with) the patient-linked sub-tokens 122, 124. Some embodiments, however, implement the transaction sub-tokens 126 as entries of a ledger table. As such, the term “blockchain” is merely an example implementation of an aspect of the ecosystem 100 and does not limit potential embodiments within the scope of this disclosure.

Similarly, a distributed ledger (e.g., blockchain 100a) may include entries of various types, such as ledger entries, blockchain blocks, and NFTs (e.g., patient tokens 120, specimen sub-tokens 122, treatment sub-tokens 124). A blockchain “token” or “block” is a discrete unit of data entered to the blockchain 100a. In the example ecosystem 100, the blockchain 100a includes a series of tokens (e.g., NFTs) as the discrete units of data as the blockchain entries.

The nodes of the ecosystem 100 generate the tokens 120, 122, 124, 126 of the blockchain 100a according to blockchain token or NFT standards (e.g., ERC-721, ERC-1155). The tokens 120, 122, 124, 126 are logically organized in a hierarchical relationship, in which a token is hierarchical “parent” token above any number of “child” tokens (sometimes referred to herein as “sub-tokens”). For instance, a patient token 120 is the hierarchical parent above a specimen sub-token 122 and a treatment sub-token 124. In an example operation, the hierarchical “parent” patient token 120 is created as an ERC-721 (or ERC-1155) token having a unique Patient Token ID (among other types of data). The hierarchical “child” sub-tokens 122, 124 are further generated as additional ERC-721 (or ERC-1155) tokens. These hierarchical child sub-tokens 122, 124 contain the Parent Token ID of the hierarchical parent, thereby establishing a logical and hierarchal link between the hierarchical child sub-tokens 122, 124 to the hierarchical parent patient token 120. In the example ecosystem 100, from the perspective of the NFT and blockchain technology and standards, the tokens 120, 122, 124 are merely tokens according to the standards (e.g., ERC-721, ERC-1155). The notional relationships of a hierarchical parent token 120 and hierarchical child tokens 122, 124 are established using the linking data, by including the Parent Token ID in each hierarchical child token 122, 124 at the time a node creates the particular hierarchical child token 122, 124.

A token contains various types of related data, such that the token represents a certain type of information relevant to the blockchain 100a. The token's data also includes various cryptographic identifiers that associate the token with blockchain 100a. For example, the specimen sub-token 122 includes the hash of the patient token 120 and another hash unique to the specimen sub-token 122, and the treatment sub-token 124 includes the hash of the specimen sub-token 122 and another hash unique to the treatment sub-token 124. This is merely an example and not limiting on potential data values or algorithms for the tokens. NFTs (sometimes referred to as “tokens” herein) are a type of blockchain data unit entry that includes more uniquely identifying data than other types of blockchain entries (e.g., ordinary blockchain blocks), typically requiring more computationally expensive algorithmic operations to generate. The NFTs often represent unique types of information and/or unique off-chain assets because unique data entries, unique identifiers, and/or unique off-chain asset render the NFTs non-fungible—not freely interchangeable. As a simple example, the specimen sub-token 122 and the treatment sub-token 124 may include the specimen data and the treatment data that are in the form of pointers to storage memory locations containing actual values of the specimen data and the treatment data. The specimen sub-token 122 and the treatment sub-token 124 are not entirely non-fungible. Similarly, the blockchain 100a may contain a plurality of specimen sub-tokens 122, each with pointers to respective off-chain storage memory locations containing the respective values for the specimen data, so the specimen sub-tokens 122 are relatively interchangeable. In these examples, it may be unnecessarily complex and expensive to generate these sub-tokens 122, 124 as NFTs. On the other hand, the patient token 120 represents a human being and, therefore, must be unique. The patient token 120 contains, for example, unique identifier information for the patient token 120 and unique information about the patient. The patient token 120 may be generated using algorithms that take multiple cryptographic values (e.g., encryption key, digital signature, digital certificate, salt, seed) and unique patient data to output the one or more unique identifiers. The patient token 120 may also contain the unique patient data corresponding to the unique human, furthering the uniqueness of this particular entry to the blockchain 100a. The patient token 120 is not feasibly interchangeable or exchangeable with another patient token (not shown) in the blockchain 100a or another blockchain (not shown). Nevertheless, the terms “token” and “sub-block” merely refer to examples of entries of the blockchain 100a and do not limit potential embodiments. For example, the patient token 120 may be a typical blockchain block, rather than an NFT (as in the ecosystem 100). As another example, the specimen sub-token 122 may be a typical blockchain block, rather than an NFT (as in the ecosystem 100).

For ease of description, the blockchain 100a of the example ecosystem 100 is uniquely associated with the particular patient, such that the ecosystem 100 employs blockchains 100a unique to each patient to represent the particular patient with the corresponding blockchain 100a. In some embodiments, however, the ecosystem 100 employs a single blockchain 100a (or fewer blockchains 100a) to represent a plurality of patients.

FIG. 1B shows components of the system 100b of the ecosystem 100 implementing healthcare NFT scaffolds. The system 100b includes an authority system 101, any number of client computing devices 106 (e.g., patient devices 106a, physician devices 106b, researcher devices 106c, oversight devices 106d), and a specimen broker system 107, which communicate via one or more communication networks 112, 114, 116. The authority system 101 includes an authority server 102 and an authority database 104, which communicate via one or more authority networks 114 internal to the authority system 101. The broker system 107 includes a broker server 108 and a broker database 110, which communicate via one or more broker networks 114 internal to the broker system 107. Embodiments may comprise additional or alternative components or omit certain components from those of FIG. 1B, and still fall within the scope of this disclosure. It may be common, for example, to include multiple client devices 106 and broker servers 108. Embodiments may include or otherwise implement any number of devices capable of performing the various features and tasks described herein. For example, the FIG. 1B shows the authority server 102 as a distinct computing device from the authority database 104, though a single computing device may comprise both the authority server 102 and the authority database 104.

The various components of the system 100b and of the computing infrastructures 101, 107 may be connected with each other via hardware and software components of one or more system networks 112 or internal networks 114, 116. Examples of such networks 112, 114, 116 include, but are not limited to, Local Area Network (LAN), Wireless Local Area Network (WLAN), Metropolitan Area Network (MAN), Wide Area Network (WAN), and the Internet. The communication over any particular network 112, 114, 116 may be performed in accordance with various communication protocols, such as Transmission Control Protocol and Internet Protocol (TCP/IP), User Datagram Protocol (UDP), and IEEE communication protocols, among others.

The computing infrastructures 101, 107 include the authority system 101 and the broker 107, each of which comprises any number of computing devices (e.g., authority server 102, broker server 108), non-transitory machine readable storage (e.g., authority database, broker database 110), and one or more internal computing networks 114, 116, which may be distinct from the one or more system networks 112, among other components. For ease of describing certain aspects and use cases, the system 100b of FIG. 1B includes the authority system 101 and broker system 107, though embodiments may include any number of such computing infrastructures 101, 107 of various types. In some embodiments, any number of computing devices, in a decentralized organization, may perform the features and functions performed by the computing infrastructures 101, 107 as described here, such that no such computing infrastructures 101, 107 are used.

The system 100b includes any number of computing devices that participate as nodes of a distributed ledger, such as the authority server 102, broker server 108, and the client devices 106, among others. For ease of description, the system 100b concentrates and centralizes certain features of the blockchain 100a at the authority system 101, though the amount of centralization may vary across embodiments. For each patient the authority system 101 generates the collection of tokens 120, 122, 124 for the token scaffold representing the patient, where the token scaffold may include any number of blockchain blocks or tokens 120 (e.g., NFTs) representing aspects of the patient's personal profile containing the patient personal data, treatments, or specimens, among other information related to the patient.

The authority server 102 generates the patient token 120 representing the patient, including patient personal information or basic information about the patient. The system 100b generates additional sub-tokens representing aspects of the patient's health based upon the patient token 120 or preceding sub-tokens. The tokens and sub-tokens include various types of data or pointers to data stored on a distributed ledger or other data storage format (e.g., relational database).

As an example, the system 100b generates a new NFT scaffold for the patient when the patient is born. The system 100b mints the patient token 120 representing patient personal data (e.g., name, date of birth, location of birth, family members) and one or more system identifiers, such as a unique Patient ID and Patent Token ID. The patient token 120 can include the patient personal data and/or pointers to patient personal data stored in a storage location (e.g., authority database 104). In some cases, the devices of the system 100b further generates one or more sub-tokens (e.g., specimen sub-token 122, treatment sub-token 124) representing the mother's treatment or any specimens related to the mother's treatment (e.g., placenta, blood samples), where these sub-tokens stem from, and are based upon, the mother's NFT scaffold.

A central authority service provider builds, operates, and/or manages the authority system 101, which performs any number of administrative roles for the system 100b. The authority system 101 functions as, for example, a source of software source related to the system 100b, a “source of truth” for information of one or more distributed ledgers, and an administrator outlet managing components of the system 100b, among any number of additional or alterative roles.

The authority service further manages and vets one or more certificate authorities that issue and validate cryptographic certificates or signatures for users of the system 100b. In FIG. 1B, the authority server 102 of the authority system 101 executes certificate authority software to issue, evaluate, and revoke certificates; and the authority database 104 stores certificates, cryptographic keys, and other information related to the certificates. Other embodiments, however, may include any number of certificate authorities hosted by any number of computing devices.

The authority server 102 executes software programming related to managing the distributed ledger and hosting the various services described herein. The authority server 102 includes any computing device having computing hardware (e.g., processors, non-transitory memory) and software for performing the various processes and tasks described herein. Non-limiting examples of the authority server 102 include servers, desktops, laptops, tablets, and the like. FIG. 1B shows only one computing device as the authority server 102, though the authority server 102 could include any number of computing devices.

The authority server 102 hosts a cloud-based app for managing operations of the system 100b. In some cases, the cloud-based app is entirely web-based (e.g., web portal), whereby the computing devices of the system 100b access the cloud-based app through a web browser. In such cases, the authority server 102 (or other computing device of the authority system 101) executes webserver software for hosting an accompanying website. Additionally or alternatively, the authority service publishes the cloud-based app as software for local installation and execution at the computing devices of the system 100b. The computing devices of the system 100b execute the app locally, which accesses the services hosted by the authority server 102. In operation, the client device 106 executes the cloud-based app (e.g., browser, locally installed software program) to access the authority server 102, which presents a user interface to an end-user and enables the end-user to interact with the various features of the system 100b. In some embodiments, the authority system 101 restricts or tailors the available features or the user interface configurations according to the types of users, such that the available features correspond to each user's role. The features available to patients, for instance, will differ from the features available to physicians. Non-limiting examples of the types of users include patients (e.g., general public), physicians or healthcare providers, researchers, biobanks, and ethics reviewers, among others.

As mentioned, the authority server 102 functions as the certificate authority. In this capacity, the authority server 102 issues, evaluates, and revokes certificates or encryption keys for end-users or entities. The authority server 102 (or other device of the system 100b) may further execute software for performing authentication operations to authenticate the users and detect the user roles based upon user login credentials or certificates, used in addition or as an alternative to the login credentials. The authority server 102 issues each end-user a certificate or set of cryptographic keys, later employed by the users and referenced by the authority server 102 when performing various operations associated with the patient's tokens and sub-tokens.

As an example, when generating a new token or sub-token, the authority server 102 receives the relevant data from the client device 106 and stores the relevant data into a particular non-transitory storage location for non-validated data, sometimes referred to as a “buffer” or “temporary storage.” The authority server 102 then evaluates one or more certificates for one or more corresponding validating users (e.g., physicians, nurses, patient). If the authority server 102 successfully validates a threshold number of certificates, then the authority server 102 generates the new token or sub-token using the relevant data previously stored in the temporary storage as non-validated data.

In some implementations, the authority server 102 requires a different threshold-number of validations based upon the type of data associated with the new token or sub-token. For example, the authority server 102 requires more validations when generating a new patient token 120 representing a new person (for an entirely new scaffold) compared to generating a new treatment sub-token 124 representing a regular checkup visit or annual physical. As another example, researchers conducting research using a patient's specimen ultimately generate certain specimen research data. Using a research device 106c, the researchers send the research data and one or more authenticating certificates to the authority server 102. In this example, the authority server 102 requires two validating users (e.g., two researchers) to upload corresponding certificates before generating the new sub-token 122 for the specimen research data. In some cases, the treatment sub-token 124 or other type of token or sub-token includes relationship data representing the relationship between the users, such as therapeutic relationship data indicating a therapeutic relationship between the physician and the patient. The relationship data of the particular sub-token may include various information or details about the relationship between the users, and include various identifiers referenced by the devices of the system, such as the user identifiers (e.g., unique patient identifier, unique physician identifier) and token identifiers (e.g., unique patient token identifier, unique physician token identifier). In this way, a patient may, for example, “claim” a treatment token or any token representing the relationship with the physician, or vice-versa, and both may have this process augmented with third party validating users (e.g., admins, colleagues, staff, family members, oversight users).

Additionally or alternatively, the authority server 102 applies a consent threshold when patient consent is required to perform various operations or transactions. The consent threshold requires the patient's certificate before the authority server 102 performs the particular operation or transaction. For example, the authority system 101 requires patient consent whenever the authority server 102 receives a custody transfer request for a specimen from a broker system 107, researcher device 106c, or other client device 106 of the system 100b. In the example, the authority server 102 executes the transaction on the blockchain 100a to reflect the change of custody only after receiving and validating the patient's certificate from the patient device 106a, though the authority server 102 may further require additional validating certificates from additional validating users. Other types of operations or transactions may also require patient consent, as represented by the authority server 102 or other device of the system 100b receiving and validating the patient's certificate.

The authority server 102 issues cryptographic data (e.g., public-private key-pairs, digital certificates, digital signatures) to stakeholder-users participating in the system 100b. The certificate authority of the authority server 102 generates and issues the new cryptographic data to the particular user according to typical processes employed in public-key infrastructures (PKIs), where the certificate authority issues the public-private asymmetric key-pair including the new signature and new certificate, which are based on the newly issued keys and the issuer signature of the certificate authority. The authority server 102 may generate the stakeholder's unique identifier (e.g., patient unique identifier) based at least in part on the new certificate, such that the nodes having appropriate access privileges may algorithmically derive a patient's unique identifier from a given token 120, 122, 124 using the patient's certificate and/or additional secret cryptographic data. Alternatively, the new stakeholder unique identifier is the new digital certificate and/or new digital signature.

The authority server 102 executes revalidation and revocation operations at predetermined periodic or random intervals. For example, when the patient expires, the certificate authority revokes the patient certificate and the patient's unique identifier expires, which propagates to each certificate or identifier of the sub-tokens 122, 124 in the patient's token scaffold. As another example, when a physician no longer holds a valid license to practice, the authority server 102 may revoke the physician's certificate and/or other cryptographic data, thus inhibiting the physician from continuing to access data, perform operations, interact with the blockchain 100a, or otherwise participate as a stakeholder in the system 100b.

The authority server 102, or other computing device node (e.g., client device 106, broker server 108) of the system 100b, generates the tokens or sub-tokens according to the various types of data received from other computing devices of the system 100b participating as nodes of the blockchain 100a, such as the client devices 106 or broker server 108. The authority server 102 receives the various types of data for the new token or sub-token. The authority server 102 generates the new token or sub-token using one or more identifiers, such as unique identifiers for the particular dataset (e.g., specimen identifier, research results identifier) and/or unique identifiers for the stakeholders involved in the operation (e.g., patient identifier, physician identifier, researcher identifier). The unique identifiers include, for example, the Patient Token ID uniquely identifies the patient token 120, and links the patient token 120 as a hierarchical parent to the various hierarchical child tokens (e.g., specimen sub-token 122, treatment sub-token 124) associated with the patient scaffold.

As mentioned, the embodiment of FIG. 1B describes the operations of managing and updating the blockchain 100a as functions performed by the authority server 102 for ease of description and understanding. Embodiments, however, need not be as structured or localized. Embodiments may include any number of computing devices of the system 100b that generate the tokens, the sub-tokens, or the updates to the blockchain 100a, provided such computing devices are participating nodes of the blockchain 100a. In some instances, the authority server 102 manages and updates transaction information for tracking custody of specimens. A researcher or other user of the system 100b receives custody of the specimens to perform clinical tests or research. The authority server 102 or other device of the system 100b updates the transaction information of the blockchain 100a information to reflect updates to the custody. For instance, the analytics server 102 or the broker server 108 generates a transaction sub-token 126 containing transaction data for the custody transaction.

The authority database 104 stores various types of data or information used for performing the processes and tasks described herein. The authority database 104 is hosted by any computing device having computing hardware (e.g., processors, non-transitory memory) and software capable of hosting the information and executing queries received from the authority server 102 or other components of the system 100b. The data stored by the authority database 104 may include patient data, specimen data, research data, researcher data, physician data, and the like, though some or all of the data may be stored in tokens, sub-tokens, or other types of entries of the blockchain 100a on distributed nodes participating as the nodes of the blockchain 100a (e.g., client devices 106). The authority database 104 stores instances of the blockchain 100a and data as a “source of truth” for the system 100b. The authority database 104 stores the data associated with tokens and sub-tokens pointed to by the tokens, sub-tokens, or other forms of data entries of a distributed ledger (e.g., blockchain 100a).

A trusted broker service operates or otherwise manages the broker system 107. The trusted broker service serves as a clearinghouse for physical specimens responsible for maintain accurate, secure, and ethical records about the specimens. The components of the broker system 107 serve as the data clearinghouse for the specimen sub-tokens 122, transaction sub-tokens 126, smart contracts, conventional databases, or other types of data related to monitoring and tracking specimen data.

The broker server 108 executes software programming related to managing the blockchain 100a and hosting the various services described herein. The broker server 108 includes any computing device having computing hardware (e.g., processors, non-transitory memory) and software for performing the various processes and tasks described herein. Non-limiting examples of the broker server 108 include servers, desktops, laptops, tablets, and the like. FIG. 1B shows only one computing device as the broker server 108, though the broker server 108 could include any number of computing devices. The broker server 108 may perform many of the previously discussed operations related managing and updating the blockchain 100a and such operations need not be repeated here.

In the system 100b, the broker server 108 may receive requests for specimens from the client devices 106 (e.g., researcher device 106c), in addition or as alternative to the authority server 102. The broker server 108 or the authority server 102 may further execute any number matching algorithms for determining suggested matches between researcher studies and cohorts of specimens or patients based on attributes common or frequently interrelated. As with the authority server 102, the broker server 108 may execute the software programming installed on the broker server 108 or retrieved from smart contracts of the blockchain 100a.

The broker server 110 stores various types of data or information used for performing the processes and tasks described herein. The broker server 110 is hosted by any computing device having computing hardware (e.g., processors, non-transitory memory) and software capable of hosting the information and executing queries received from the broker server 108 or other components of the system 100b. The data stored by the broker server 110 may include patient data, specimen data, research data, or researcher data, though some or all of the data may be stored in tokens or sub-tokens of the blockchain 100a, or any other form of entry of a distributed ledger (e.g., ledger table), on one or more participating nodes of the blockchain 100a (e.g., client devices 106).

The client devices 106 function as nodes of the blockchain 100a. The client devices 106 includes any computing device having computing hardware (e.g., processors, non-transitory memory) and software for performing the various processes and tasks described herein. Non-limiting examples of the client devices 106 include servers, desktops, laptops, tablets, and the like.

In some embodiments, the client devices 106 have various configurations based upon the roles of the intended users. For example, the patient device 106a may access certain features of the cloud-app that are different from the features accessed by the physician device 106b or researcher device 106c.

In some embodiments, the client devices 106 comprise one or more peripheral interfaces for receiving validating inputs from receives. Non-limiting examples of the types of interfaces may include USB, PCI, RFID, NFC, or other technology for inputting validating inputs (e.g., digital certificate, digital signature) into a workstation using a physical device. In operation, the client device 106 transmits the validating input to other nodes of the system 100b to validate certain transaction operation or authenticate the particular user. Alternatively, the client device 106 executes the particular transaction operation locally and, as such, executes the particular validation or authentication operation locally using the validation input received from the user.

### Example Process Operations

### Initializing New Token Scaffold, and Generating New Token and Sub-Token

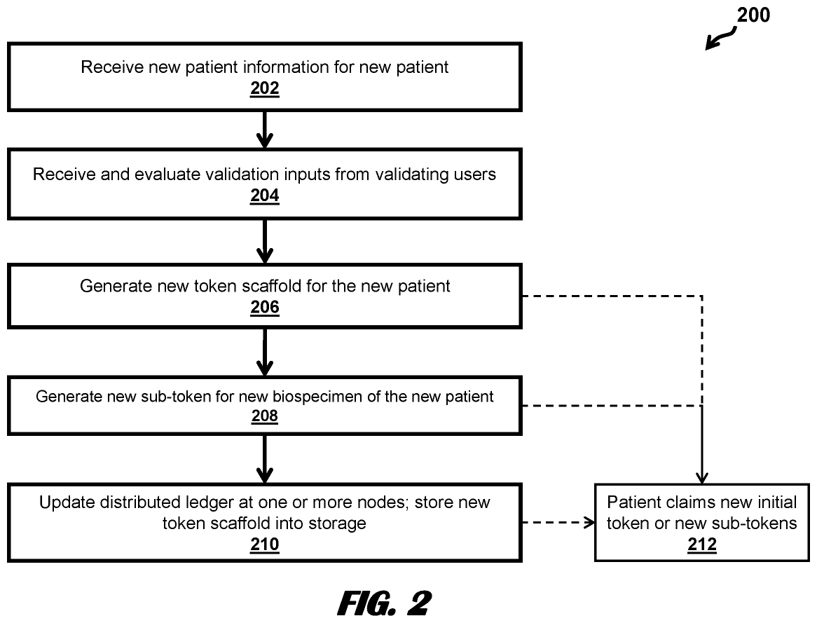

FIG. 2 shows execution steps of a method 200 for initializing a new token scaffold for a new patient. Embodiments may include additional, fewer, or different operations than those described in the method 200. The method 200 is performed by a server executing machine-readable software code associated with a distributed ledger ecosystem of participating nodes, though it should be appreciated that one or more computing devices or processors may perform the various operations described in FIG. 2. The example method 200 implements a blockchain having blockchain tokens (e.g., NFTs) according to NFT standards (e.g., ERC-721, ERC-1155), such that nodes participating in the blockchain generate the tokens for the blockchain in accordance with the NFT standards (e.g., ERC-721, ERC-1155). The token may be logically organized in a hierarchical relationship, in which a token is hierarchical “parent” token above any number of “child” tokens (sometimes referred to herein as “sub-tokens”). For instance, a patient token is a hierarchical parent above a specimen sub-token and a treatment sub-token. The notional relationships of a hierarchical parent token and hierarchical child tokens may be established by including linking data (e.g., a Parent Token ID that uniquely identifies the hierarchical-parent patient token) in each hierarchical child token when a node creates the particular hierarchical child token. As an example, the node creates the patient token as an ERC-721 (or ERC-1155) token having the unique Patient Token ID (among other types of data). The same or different node creates a hierarchical child sub-token as a new ERC-721 (or ERC-1155) token. The new hierarchical child sub-token contains the Parent Token ID of the patient token, thereby establishing the logical and hierarchal link between the patient token (as the hierarchical parent) of the sub-token (as the hierarchical child).

In step 202, the server receives new patient information for a new patient from a physician device or other computing device. A physician inputs the new patient information into a graphical user interface (GUI) of the physician device, which the server receives as various types of data, such as patient personal data to form a patient profile. The server or the physician device may encrypt some or all of the data, before or while generating an initial token for the token scaffold.

Non-limiting examples of the patient personal data for the patient profile data may include: a patient name; date, time and location of birth; biometric data and identifiers (e.g., fingerprints); unique genetic data (e.g., some or all of the genome sequence); omic data (e.g., proteome, microbiome social mediome); identity of parents, including biological, birth parents and guardians; health status (e.g., living, deceased, breast cancer, hypertension); proxies or other representatives; physician identities; designated or elected stewards of data or property and nominees information; locations associated with the patient (e.g., home address(es), city/state); patient identifiers (e.g., state ID, national ID, international ID, SSN, driver's license, passport number); medical record identifiers from each health system/provider where they receive care and other customer identification numbers (e.g., assigned by direct to consumer genetics or ancestry companies); and current specimen sub-block inventory (or sub-token inventory), among others. A physician (or healthcare provider) token may similarly include physician profile data, such as location data for one or more sites of practice, along with other identifying profile information about the physician or practice.

In step 204, the server receives and evaluates validation inputs purportedly received from validating users. The validating user may include physicians or other healthcare workers, patients, administrators, researchers, or other type of user who uses the blockchain ecosystem. The validation input may include any type of validating data (or a pointer to the validating data) associated with a particular validating user and indicates the validating user's acquiescence of adding data to the distributed ledger. Non-limiting examples of the validating data may include a cryptographic certificate or signature, authentication credentials, or the like. The server requires a threshold number of validation inputs before performing

The validating data may be stored on any computing device or electronic storage device accessible to the validating user or the server. For example, the validating data includes a certificate stored on a portable storage device, such as a handheld USB storage drive, which the validating user inserts into a USB interface of the client device. When generating the token, the server prompts the validating user to upload and transmit the certificate to the server.

As another example, a storage server stores the certificates of a plurality of validating users. In this example, when generating the new token, the validating user enters authentication credentials for accessing the validating user's certificate. If the storage server successfully authenticates the purported authentication credentials, then the storage server accesses and transmits the certificate to the server.

The validating users enter the new patient information into the GUI interface of the physician device and the validating inputs. The server receives the new patient information as patient data, which the server stores into a buffer memory location while awaiting the validating inputs and evaluating the validating data. Each validating user (e.g., physician, nurse, patient, proxy of the patient) enters a corresponding validation input into the physician device (or other computing device), which the physician device transmits as the validation data to the server. Each member of the healthcare team (e.g., physician, nurses) may take turns connecting a corresponding USB storage device containing that particular validating user's certificate. The physician device transmits these certificates to the server. The patient or patient's proxy may also input the certificate of the patient or patient proxy using the physician device or other computing device. In some circumstances, however, the patient or proxy may access a cloud-based app configured to receive the patient's or the proxy's authentication credentials. If the cloud-based app authenticates the authentication credentials, then the server receives or accesses the certificate of the patient or proxy.

In step 206, if the server received the threshold number of validating inputs (in step 204), the server initializes the token scaffold for the new patient and generates a new initial token for the patient. The server generates the new initial token using the various types of patient data previously stored in the buffer memory. The initial token includes

If the server fails to receive and validate the threshold number of validating inputs (in step 204), then the server may continue to store the data in the buffer memory for a predetermined or indefinite amount of time in accordance with a retention configuration.

In step 208, the server generates one or more new sub-tokens representing additional types of data received (simultaneously or contemporaneously) with the patient data. The additional types of data may include, for example, specimen data corresponding to one or more specimens for the patient, though additional types of data may be received for generating the one or more new sub-tokens. For example, at the time of the patient's birth the healthcare workers enter specimen data for various types of specimens taken during labor treatment (e.g., placenta, umbilical cord segment, cord blood, amniotic fluid sample). The server or other device generates sub-tokens for the respective specimens, where the specimen sub-tokens represent the specimen data for the respective specimen. These sub-tokens, representing each of the specimens associated with the patient's birth, become the first set of sub-tokens to populate the patient's token scaffold. In addition, the specimen data may be included into device-generated sub-tokens appended to the mother's token scaffold.

The server may require further (or renewed) validating inputs from the validating users, though not in each configuration or embodiment. In some cases, the server may forgo the additional validation inputs to generate the new sub-tokens, if the server received the additional types of data (e.g., specimen data) in conjunction with the patient profile data (or other types of data). In such cases, the server reuses the validation data previously received to generate the new initial token (in step 204). Additionally or alternatively, in some cases, the buffer storage receives and stores the additional types of data for the new sub-token, such that the server may evaluate the validating inputs (as in step 204) for the data of the initial token simultaneously or contemporaneously with the additional data for the sub-token.

In some embodiments, the server (or other device of the system) dynamically queries a database of specimens or specimen sub-tokens having similar specimen attributes, based upon comparing the specimen data of the new sub-token against various types of data in extant patient tokens or specimen sub-tokens in the ecosystem. The tokens or sub-tokens include unique patient identifiers that enable to server to dynamically associate, or store predetermined associations, between the patient and other patients in a common set or cohort of patients having similar attributes (e.g., patients with similar conditions, similar circumstances, similar features, biomarkers, common familial relations).

In step 210, the server updates the distributed ledger at one or more nodes, and stores the new initial token and sub-tokens into one or more storage locations. The server updates the distributed ledger to indicate various transactions, including the indicating the generation of the new token and sub-tokens. The server or other computing device (e.g., client device) includes NFT wallet software and a non-transitory storage medium configured to store the new token and sub-tokens of the patient's scaffold.

In optional step 212, the patient “claims” the initial token or sub-token, whereby the server or other computing device associates the initial token or sub-tokens with the patient's token scaffold. The users and devices associated with the method 200 may execute the operations for claiming the token or sub-token at one or more moments or at the conclusion of the method 200. For example, the patient may perform the operations for claiming the initial token after the server generates the initial token (in step 206). As another example, the patient collectively claims the initial token and the sub-tokens after the server and any participating nodes update the distributed ledger (in step 210).

The patient receives a notification (e.g., email, text message, UI prompt) indicating that the server (or other node) generated a particular new token (e.g., initial token, sub-token). Notification indicates to the patient that the new token is purportedly associated with the patient and available for the patient to claim. The validating user responsible for generating the new token may manually indicate to the server that the new token is associated with the patient by entering a patient identifier or contact information for the patient. Alternatively, the server may automatically determine that the new token is associated with the patient based upon one or more identifiers of the new token.

To claim the particular new token (e.g., initial token, sub-token), the patient accesses the cloud-app and selects the new token from UI displaying a claim queue. In some embodiments, the server (or other device hosting the cloud-app) permits the patient to claim the new token without further inputs, whereby the server relies upon the patient's successful authentication during a login routine to access the cloud-app. In some embodiments, however, the server requires one or more validating inputs from the patient before permitting the patient to claim the new token. The server, for example, may require the patient to upload or otherwise transmit the patient's certificate to the server. In some cases, the server permits the patient to claim the new token when the server successfully validates the certificate received from the patient device (or other device). In some cases, the server generated the new token based, in part, upon one or more cryptographic algorithms using one or more identifiers of the patient. The server performs one or more cryptographic algorithms to derive a certain predicted value using the data of the new token and the purported certificate received during the claiming operation. Additionally or alternatively, the server issues a claim ticket as a type of validation data associated with the particular new token. The patient enters or transmits the claim ticket to the server a validating input, in addition or as an alternative to the patient's certificate. As before, the server performs the one or more cryptographic algorithms to derive the predicted value using the data of the new token, the claim ticket, and/or the purported certificate. If the server determines that the predicted value derived using the claim ticket and/or the purported certificate matches an expected value, then the server permits the patient to claim the new token and associates the new token with the patient's scaffold.

In some circumstances, the patient's proxy (e.g., newborn's mother) claims the initial token or the sub-token on behalf of the patient (e.g., newborn).

In some implementations, the patient may claim all of the patient's specimen tokens once the specimen tokens enter the system. For instance, a biobanker in possession of a collection of biosamples and the biobanker registers a biobanker user account in the service application. The biobanker uploads the information about the de-identified biosamples to the server of the service application. The server creates a token (e.g., NFT) for each biosample. As such, the patient's real identifying information is unavailable due to the biosample information being stripped of identifying information. When a patient registers for the service in the service application, the patient provides consent to re-identify the biosamples. The service application (e.g., authority server, patient device) transmits the consent input, provided via a consent user interface, to a computing device of the biobank by the service application. Based on the consent provided by the patient, the biobanker's computing device automatically or manually (by user inputs) reveals identifying information of the biosamples associated with the patient. Thereafter the service application can directly link the patient's real identifying information with the patient's biosamples. The features for claiming or managing biosamples may be compatible with both custodial and non-custodial approaches. The patient need not control the account that manages the patient's biosamples in order for the patient to be considered the “owner” of the biosamples.

### Biobank and Researcher Accessing Portion of a Token Scaffold

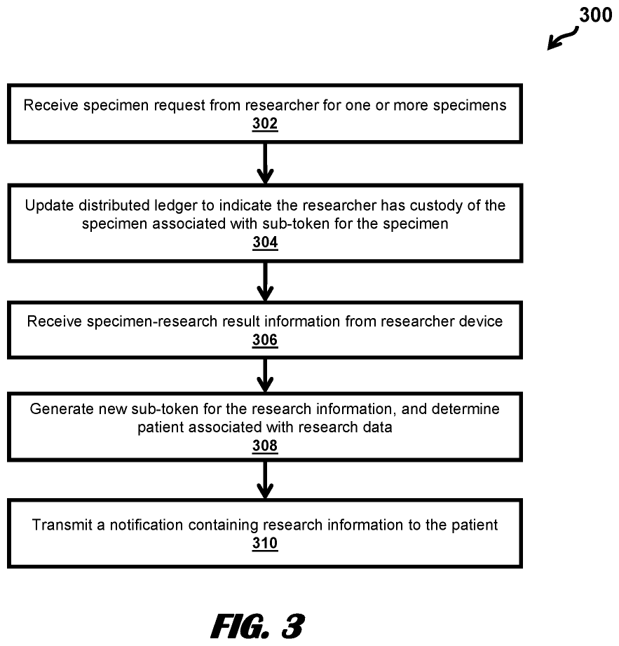

FIG. 3 shows execution steps of a method 300 for third-party (e.g., broker service, researchers, oversight users) access to a portion of a token scaffold and updates to the token scaffold. Embodiments may include additional, fewer, or different operations than those described in the method 300. The method 300 is performed by a server executing machine-readable software code associated with a distributed ledger ecosystem of participating nodes, though it should be appreciated that one or more computing devices or processors may perform the various operations described in FIG. 3. The server, for example, may include the authority server or the broker server described in FIG. 1B. The example method 300 implements a blockchain having blockchain tokens (e.g., NFTs) according to NFT standards (e.g., ERC-721, ERC-1155), such that nodes participating in the blockchain generate the tokens for the blockchain in accordance with the NFT standards (e.g., ERC-721, ERC-1155). The token may be logically organized in a hierarchical relationship, in which a token is hierarchical “parent” token above any number of “child” tokens (sometimes referred to herein as “sub-tokens”). For instance, a patient token is a hierarchical parent above a specimen sub-token and a treatment sub-token. The notional relationships of a hierarchical parent token and hierarchical child tokens may be established by including linking data (e.g., a Parent Token ID that uniquely identifies the hierarchical-parent patient token) in each hierarchical child token when a node creates the particular hierarchical child token. As an example, the node creates the patient token as an ERC-721 (or ERC-1155) token having the unique Patient Token ID (among other types of data). The same or different node creates a hierarchical child sub-token as a new ERC-721 (or ERC-1155) token. The new hierarchical child sub-token contains the Parent Token ID of the patient token, thereby establishing the logical and hierarchal link between the patient token (as the hierarchical parent) of the sub-token (as the hierarchical child).

Certain embodiments described herein, such as the example method 300, involve on-chain or blockchain-related operations. However, in some embodiments, the operations may reduce or minimize the interactions between computing devices with the blockchain as necessary. While the biospecimen and most of the content related to the biospecimen data will be referenced on-chain, in some embodiments, the actual biospecimen data itself can be maintained off-chain, in non-transitory storage media of a centralized database. The database stores, off-chain, various types of information, such as user information and biosample information, on the private backend centralized storage. The database is accessed when presenting the types of data accessed by a client device on a frontend user interface of user application. The database serves as a storage of data off-chain within a service application and maintained privately by a host authority service that hosts the client application. The database stores the data about, for example, the patient profile, researchers, and the biosamples in the system. The database stores the possession or ownership associated with the biosamples, and the same or similar ownership information about the biosample is captured as biospecimen data on one or more types of NFTs of the blockchain. The NFTs on the public blockchain are immutable. The off-chain storage helps manage the private information about the patients and patient biosamples within the centralized database of the service application without making such private information available on the public blockchain. In such embodiments, the system and method implements a hybrid approach, including both on-chain and off-chain data storage components. Some types of information will be maintained on-chain, while the rest of the information will be managed off-chain, in the centralized database. The portions of on-chain information may include pointers or other types of information, such as unique identifiers, that reference the stored data in the database.

In step 302, the server receives a specimen request for one or more specimens from a computing device of a researcher-user. These will be programmable functions executed by the server or the computing device, which may include data translation and/or data validation operations related to the clinical scenario via an application programming interface (API) program. The server maintains a database of available specimens and corresponding sub-tokens associated with any number of patients. The specimen request indicates one or more specimen attributes desired by the researcher, such as the type of specimen and attributes of the specimen patient-donor, among others. The server determines a set of one or more specimens that satisfy the parameters of the specimen request and the broker sends the specimens to the researcher. The server updates the distributed ledger to indicate a custody transfer transaction associated with each specimen sub-token. In some embodiments, an NFT wallet of the server or the patient-donor maintains custody of the particular specimen sub-tokens. In some embodiments, an NFT wallet of the researcher receives the specimen sub-tokens.

In some embodiments, the server automatically generates the specimen request based upon one or more machine-learning or matching algorithms. In such embodiments, the server executes pre-configured algorithms that continually or periodically identify specimens satisfying pre-configured research request parameters. For example, the tokens or sub-tokens include unique patient identifiers that enable to server to dynamically associate, or store predetermined associations, between the patient and other patients in a common set or cohort of patients having similar attributes (e.g., patients with similar conditions, similar circumstances, similar features, biomarkers, common familial relations).

The specimen data captured in the specimen sub-tokens is organized and marked according to a data structure corresponding to one or more anatomic, physiologic, and/or pathologic taxonomies. The taxonomy data structure allows the server or other device to query the specimen information related to a given type of specimen for a patient according to the research request. The server may identify other specimen sub-tokens for patients having similar specimen data. The patient, proxy, or other representative (e.g., parent, physician) may perform certain management operations for managing data distribution and consent configurations on behalf of the patient via a configuration UI of a cloud-app. The particular user may perform these management operations at any time, often before the server performs the query according to the researcher request. The server or other device maintains the data structures of the taxonomies transparently on the blockchain. The library of taxonomy or a particular taxonomy grows over time, as the server or a user discovers and configures additional categories.

In some embodiments, smart contracts implemented on the blockchain instruct the server or other nodes to observe the various types of data corresponding to the NFTs and execute a classifier algorithm to classify the data of the NFTs or the NFTs into various cohorts based on common attributes, such as health conditions or biomarkers, among others. The server or storage media uniquely label each cohort and dynamically updates a cohort membership listing of patient identifiers (or other type of identifier) as the cohort membership changes over time. The server may store the membership listing and/or the membership attributes transparently on the blockchain and/or in a particular database.

Although the researchers receive the specimens, specimen data, and/or the specimen sub-tokens, the researcher device and the researcher do not receive, and cannot access, unique patient identifiers or any personally identifying information of the specimen donors. The researcher device, however, may access specimen data in the sub-token, including specimen attribute information, a unique specimen identifier, and a hash or otherwise obfuscated (e.g., encrypted) version of a patient identifier. The researcher device does not have access to certain secret information (e.g., cryptographic certificate, encryption key, cryptographic seed, cryptographic salt) required to algorithmically recover the patient identifier from the specimen sub-token in a plaintext, readable format. The server or other client devices (e.g., patient device, physician device), on the other hand, stores the secret information or may request and receive access to the secret information. In this way, the research device does not have access to the personally identifying information or any other token or sub-token of the patient scaffolds. The researcher device's access is limited to the particular specimen data.

In step 304, the server updates the distributed ledger to indicate that the researcher has custody of the specimen associated with sub-token for the specimen. The server or other device executes software programming of the server or a smart contract of the blockchain to update the distributed ledger in accordance with one or more researcher-user instructions, patient-user configurations, and/or validating inputs. The server and nodes of the ecosystem maintain transparency regarding movement of specimens and specimen data along the supply chain of users or entities. The nodes maintain consensus across nodes in the supply chain regarding the “state of truth” as various types of transaction data are added over time.

Similar to user validation operations discussed above, in some embodiments, the server requires a threshold number of validating inputs from the researcher-users prior to executing the specimen-custody update. In such embodiments, the server receives the one or more validating inputs from the researcher device in conjunction with receiving the specimen request (in step 302). The server evaluates the validation data (e.g., certificate, credentials) of the validating inputs to determine whether the validation data satisfactorily validates the corresponding researcher-users. If the server validates the threshold number of validating inputs, then the server executes the custody-update transaction, and the broker service sends the physical specimen to the researcher.

Additionally or alternatively, the server requires the validating input for the patient, thereby representing consent for the particular transaction. In some cases, the patient preconfigures, via a cloud-app UI, the server or a smart contract with preconfigured consent for particular cohorts, types of research, or researchers. In some cases, the server or other device executes smart contracts of the blockchain associated with the NFTs (e.g., patient token, sub-tokens). These smart contracts include instructions for the server to implement, evaluate, and gather individual consent or collective consent in a cohort. For instance, the server executes a smart contract for determining whether the patient consent is required and request patient consent (e.g., validating input) if needed. In some embodiments, patient consent for the particular specimen is facilitated when the server executes a smart contract that transparently implements a voting process for nodes associated with each NFT of a cohort of NFTs sharing common attributes. The patient may also configure the server to preclude custody transfer transactions, thereby opting out of some or all specimen studies based upon the patient's configurations.

In step 306, the server receives new specimen-research result information from a researcher device. The researchers conduct various research efforts using the specimens of corresponding patient-donors. While conducting the research, the researchers input results data indicating research findings in various computing programs and/or the laboratory or clinical devices generate various types of results data corresponding to the researcher findings. For example, the research results data may represent organoids, lab device outputs, word processing data, spreadsheet data, or any other form of data representing research result information associated with a particular specimen. The researcher inputs, converts, transmits, or otherwise provides the results data to the researcher device.

In step 308, the server generates a new sub-token representing the research results information. The server further determines the particular patient and patient scaffold associated with the new research results data for a particular specimen. The researcher device transmits the research results data to the server and a request for server to generate the new research results sub-token. The server generates this new research results sub-token based upon the research results data and using one or more identifiers associated with the patient scaffold, the patient token (e.g., patient identifier), and/or other sub-tokens of the patient scaffold (e.g., specimen sub-token, specimen unique identifier).

The server may appends the new research results sub-token to the patient scaffold. Alternatively, the patient accesses the cloud-app to perform a claim operation to associate (or “claim”) the new research results sub-token with patient's scaffold.

The research results data and/or the new results sub-token may include a results-data unique identifier that the server or the researcher device algorithmically generates based upon the one or more identifiers associated with patient's scaffold. An aspect of the embodiments described herein includes software programming for obfuscating and de-obfuscating any patient-identifying data of the patient token or sub-tokens. In operation, the server receives the research results data in the form of the new results sub-token or in another format received from the researcher device. The server performs one or more algorithmic operations to de-obfuscate or re-identify the patient-identifying data in the research results data to determine the particular patient associated with the incoming research results data. To de-obfuscate and/or re-identify the appropriate patient unique identifier, the server applies one or more cryptographic algorithms on the results-data unique identifier and one or more cryptographic values (e.g., keys, certificates, seeds). The server or the researcher device previously calculated the results-date unique identifier from the patient unique identifier directly or indirectly using intermediate identifiers (e.g., specimen unique identifier), which enables the server to algorithmically derive the patient unique identifier. The server then associates the research results data with the patient scaffold.

The server may use additional or alternative identifiers associated with the research results data or the particular specimen to determine which patient scaffold is associated with the particular research results data and specimen. The research results data may include, for example, an identifier for the research cohort or the particular specimen, which the server uses to calculate the patient-identifying data.

In some embodiments, the researcher device generates the new research results sub-token and transmits the results sub-token to the server. As before, the server derives the patient unique identifier or the specimen identifier using the results unique identifier to determine the particular patient scaffold associated with the new sub-token received from the researcher device.

The server may require a threshold number of validating inputs for the researchers before generating the sub-token or otherwise associating the research results data with the patient scaffold. Each researcher inputs a validation input to the server, and the server evaluates validation data (e.g., certificate, authentication credentials) of each validation input to determine whether the validation data is valid. If the server successfully validates the threshold number of validating inputs, then the server generates the new sub-token.

In optional step 310, the server transmits a notification containing some or all of the research results data to the patient device. Using the patient-identifying data (derived in step 308), the server queries configuration or preferences instructions stored in a configuration database or in the patient token to determine one or more communication channels (e.g., SMS, MMS, email, telephone) for formatting and communicating the notification to the patient. This beneficially allows the patient to receive the research results information pertaining to the patient's specimen, while also maintaining the patient's privacy and obfuscating the patient's identity from the researchers.

### Physician Accessing a Token Scaffold

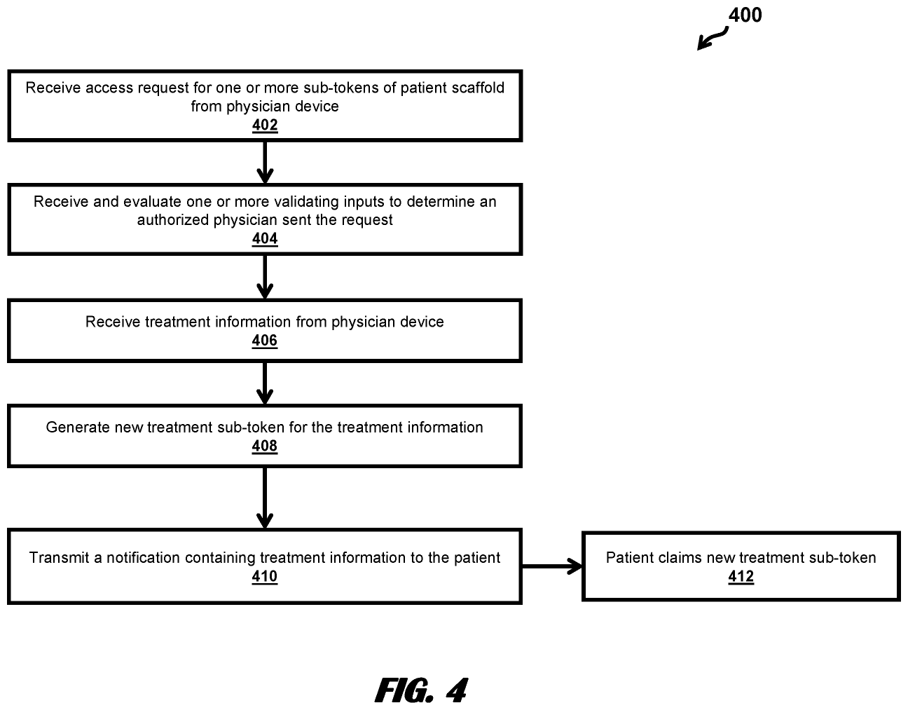

FIG. 4 shows execution steps of a method 400 for physician access to the token scaffold and updates to the token scaffold. Embodiments may include additional, fewer, or different operations than those described in the method 400. The method 400 is performed by a server executing machine-readable software code associated with a distributed ledger ecosystem of participating nodes, though it should be appreciated that one or more computing devices or processors may perform the various operations described in FIG. 4. The example method 400 implements a blockchain having blockchain tokens (e.g., NFTs) according to NFT standards (e.g., ERC-721, ERC-1155), such that nodes participating in the blockchain generate the tokens for the blockchain in accordance with the NFT standards (e.g., ERC-721, ERC-1155). The token may be logically organized in a hierarchical relationship, in which a token is hierarchical “parent” token above any number of “child” tokens (sometimes referred to herein as “sub-tokens”). For instance, a patient token is a hierarchical parent above a specimen sub-token and a treatment sub-token. The notional relationships of a hierarchical parent token and hierarchical child tokens may be established by including linking data (e.g., a Parent Token ID that uniquely identifies the hierarchical-parent patient token) in each hierarchical child token when a node creates the particular hierarchical child token. As an example, the node creates the patient token as an ERC-721 (or ERC-1155) token having the unique Patient Token ID (among other types of data). The same or different node creates a hierarchical child sub-token as a new ERC-721 (or ERC-1155) token. The new hierarchical child sub-token contains the Parent Token ID of the patient token, thereby establishing the logical and hierarchal link between the patient token (as the hierarchical parent) of the sub-token (as the hierarchical child).

Certain users of the ecosystem, such as researchers, must receive limited access to the patient's scaffold and personally identifying information about the patient. Physicians, however, may need to access a broader or full scope of patient information. When treating or reviewing records of the patient, the physician analyzes a variety of information about the patient, including current treatment issues and prior health history, and the like. The physician is likely to require various types of data represented by the patient token and a plurality of sub-tokens of the patient scaffold, such that the physician may access and analyze the all or most of the digital twin or EHR for the patient. The method 400 relates to access and update operations for the patient's token scaffold by physicians or other types of users (e.g., proxies, ethics oversight reviewers) who seek broad or complete access to the patient's scaffold and private information. The method 300 of FIG. 3 relates to access and update operations by researchers or other types of users (e.g., insurers, employers) who require comparatively limited access to the patient's scaffold and private information.

In step 402, the server receives an access request for one or more sub-tokens of a patient scaffold from a physician device, where the access request may be manually or automatically generated according to inputs from the physician. The physician device executes accesses a cloud-app for participating nodes of the ecosystem and hosted by the server or other device. The access request indicates, for example, the patient identifier for the patient scaffold. The access request may also indicate the types of data or sub-tokens sought by the physician, which the server uses to retrieve the appropriate types of data or sub-tokens according to a taxonomy data structure.

In some embodiments, the server executes one or more matching algorithms to determine and suggest a physician-to-patient pairing and dynamically generates the physician request based upon the pairing. The server queries physician data for physicians registered with the ecosystem, where the physician data is stored in a database or blockchain entries. The server identifies a suggested physician based upon matched attributes indicated by the taxonomy data structure. For example, the server determines from a particular sub-token of the patient's scaffold that the patient has a particular health issue, and identifies a physician specializing in that particular health issue. The server may grant access to the physician or otherwise associate the physician data with the patient's scaffold, if the patient approves the suggested association.

In step 404, when the physician attempts to access data of the patient's scaffold, the server receives and evaluates one or more validating inputs to determine whether the authorized physician-user originated the access request. As discussed, the validating inputs may include the authentication credentials or cryptographic certificate of the physician. The server or other device (e.g., authentication server, active directory server, certificate authority server) evaluates the validation data of the validating input to authenticate the physician. If the server determines an authorized physician originated the access request, then the server grants the physician device access to some or all of the data available in the patient's scaffold. In some embodiments, the server retrieves, decrypts, and presents to the physician various types of data represented by the token and sub-tokens via a GUI of the physician device. Alternatively, the server transmits to the physician device a cryptographic key or value that enables the physician device to access, retrieve, decrypt, and present the various types data represented by the token and sub-tokens.

In some cases, the patient submits preference configurations to the server via a patient device configuring certain instructions or operations of the server or other devices in accordance with the patient's preferences. The preferences may indicate the one or more physicians or physician unique identifiers permitted to access the patient's token scaffold. In some cases, the server transmits an access notification to the patient device via one or more communication channels, where the access notification requests the patient approve or deny the access request that the server received from the physician device.

In some circumstances, the patient is unfamiliar with the physician and/or unable approve the physician's access request. For example, when a hospital emergency room admits the patient and the patient is unconscious, incoherent, or otherwise unable to help access the patient's token scaffold. In such circumstances, the physician registers with the ecosystem and physician data is stored into a database or represented by an entry of the distributed ledger (e.g., blockchain physician block, physician token), allowing the server to query the database, distributed ledger, or participating nodes for the physician data to determine whether the physician is registered with the ecosystem as a physician or particular type of physician. The server authenticates the physician and the physician device using the physician data and the validating inputs received from the physician device. The physician data may include or direct the server to expected validation data (e.g., authentication credentials, cryptographic certificate data) for the physician. The server compares or otherwise validates the validation data (e.g., authentication credentials, certificate) received in the validating input using the expected validation data for the physician.

Additionally or alternatively, the server receives and evaluates validation inputs for the patient in conjunction with the physician's access request, thereby representing the patient's consent for the physician to access the patient's token scaffold.

In step 406, the server receives treatment information from the physician device. The treatment data includes one or more unique identifiers, such as a treatment unique identifier, a physician identifier, a patient identifier, and the like. The physician device encrypts some or all of these identifiers, such as the physician identifier and the patient identifier. The physician device algorithmically calculates the treatment unique identifier using one or more identifiers of the patient token or sub-token of the patient's scaffold.

In the method 400, the server receives the treatment information as treatment data in various data formats and generates a treatment sub-token using the treatment data (in step 408, below). But in some embodiments, the physician device generates the treatment sub-token, such that the server receives the treatment data represented by the new treatment sub-token.

In step 408, the server generates the new treatment sub-token representing the treatment data received from the physician device. The physician device transmits the treatment data to the server (step 406) and a request for server to generate the new treatment sub-token. The server generates this new treatment sub-token based upon the treatment data and using the one or more identifiers associated with the patient scaffold, the patient token (e.g., patient identifier), and/or other sub-tokens (or sub-blocks) of the patient scaffold.

The server may append the new treatment sub-token to the patient scaffold. Alternatively, the patient accesses the cloud-app to perform a claim operation to associate (or “claim”) the new treatment sub-token with patient's scaffold.

In some circumstances, the server may need to determine the patient scaffold associated with the new treatment sub-token. The server may algorithmically derive a patient unique identifier from the treatment data by applying one or more cryptographic algorithms on the previously calculated treatment unique identifier of the treatment data. In some circumstances, however, the physician device or the server receives access to patient-identifying information. As such, the server or physician device need not algorithmically derive the patient unique identifier or various other unique identifiers of the patient's scaffold.

In step 410, the server transmits a notification containing some or all of the treatment data to the patient device. Using known or derived patient-identifying information, the server queries preference configurations of the patient stored in a database or the patient token. Based on the preference configurations, the server determines one or more communication channels (e.g., SMS, MMS, email, telephone) for formatting and communicating the notification to the patient device.

In optional step 412, the server receives the patient claims the new treatment sub-token, associating the new treatment sub-token with the patient's scaffold. The users and devices associated with the method 400 may execute the operations for claiming new treatment sub-token at any time after the physician device generated the treatment data (in step 406) or after the server generated the new treatment sub-token (in step 408).

The patient receives the notification (in step 410) indicating that the server or other node generated the new treatment sub-token. The notification may indicate that the new treatment sub-token is purportedly associated with the patient and available for the patient to claim. The patient accesses the cloud-app and enters one or more validating inputs permitting the patient to review and claim the available treatment sub-token. In some circumstances, the patient's proxy (e.g., minor's parent) claims the treatment sub-token on behalf of the patient (e.g., minor).

### Additional Embodiments and Features

### Patient Value and Compensation for Research Results

Embodiments may implement smart contracts including a mechanism for monetizing and equitably distributing the valuable assets (e.g., specimens, research results) created during healthcare practice or research. The nodes may are immutably memorialize the various types of data described herein ensuring that healthcare benefits, personal benefits, and financial benefits are equitably or appropriately apportioned.

Embodiments may implement smart contracts including a mechanism to automate and ensure efficient translation of knowledge derived from clinical data (e.g., research results) into personalized patient care and/or monetary compensation for those patient-donors whose specimens and specimen data contributed to researcher activities. By ensuring that individual patients benefit from such research contributions, the system may beneficially address disparities inherent in a learning health system that obligates patients to contribute without guaranteeing just distribution of benefits and burdens.

As an example, some embodiments implement a unit of currency to represent a data credit for the patient-donors whose specimens contribute to research activities. The “data credit” represents the contributions of individual patients and changes during the lifecycle of the specimen when used in further research. The system precisely tracks the contributions of the specimen used in a given research study and implements the mechanism to directly reward them in proportion to their contribution. Furthermore, smart contracts implementing this agreed upon reward mechanism provide a guaranteed and equitable distribution of the valuable assets created.

### Optional Stakeholder NFTs

Embodiments may generate tokens for physicians, clinical researchers, and other stakeholders. The unique NFTs include relevant attributes for each stakeholder in the system. For instance, a physician profile represented by physician token includes name, location, license information (e.g., local certifications, state certifications, national certifications), certificate of undergraduate and graduate medical education, a list of individuals who authenticate the physician (e.g., a list of physician peers, a list of non-physician peers, a list of patients) at some interval (e.g., 6 months, 1 year, 5 years), physician well-being reports by peers, a list of peer-reported qualities, and continuing education credit status, among other types of data. In some cases, physician NFTs or sub-tokens may be claimed or minted upon receiving a medical license to practice independently (not supervised). In some cases, residents may have a form of a trainee/shadow physician NFT or sub-token that will enable the resident to remain connected to and voter's rights regarding allocation of patient specimens proportional to the resident's role in the patient care (this voting power may increase upon receiving a license to practice).

The nodes of the system may executes machine-learning classifier software that analyzes the attributes of the stakeholder tokens. For instance, a patient device executes a smart contract-based program to discover, identify, and classify or match the patient with physicians. In some instances, the system incentivizes continuity of care by rewarding the NFTs of patients who practice continuity of care.

Similarly, the system will include clinical researcher profiles in researcher tokens. The researcher profiles include researcher data such as publications, peer-review, institution affiliations, funding mechanisms, and a listing of peers, among other types of data.

### Supply Chain Oversight and Ethics Oversight

Individuals and devices involved with a supply chain typically cannot capture on the public blockchain every movement of specimens and relevant data along the supply chain. Embodiments, however, include smart contract programming for integrating and capturing on-chain and off-chain components of the movement along the supply chain. Integrity of off-chain movements are enforced through automated smart contract programming and certain human protocols (e.g., Institutional Review Board (IRB) reviews). Off-chain movement information is aggregated and recorded on-chain as transaction entries to the distributed ledger, allowing the custody transfers to be auditable and verifiable across the supply chain. The custody transfer entries on the distributed ledger provide the ability to track the specimen location and responsible parties over time.

The system may generate multi-stakeholder sub-tokens as a mechanism for ensuring accountability to ethical standards, scientific standards, and/or financial standards for the lifecycle of the specimen and for oversight of the data use. Oversight devices or other nodes participate in various oversight operations, such as IRB reviews, licensing agreement reviews, consent agreement reviews, and peer reviews. The multi-stakeholder sub-tokens are associated with smart contracts that permit the oversight devices to access the data of the particular sub-token (e.g., specimen sub-token, research sub-token) for review and provide oversight feedback, and that maintain consensus in perpetuity (e.g., timely distribution of relevant results) as additional value is added over time to the sub-token.

The token system may enables continuous, decentralized ethical surveillance of how stakeholders use a specimen, via a distributed collection of patients, physicians, university researchers, biotechnology industry professionals, and other stakeholders (e.g., IRB members, honest brokers, research funders), and allows dynamic engagement and response to feedback. Oversight users may access the data of certain sub-tokens to review and verify the accuracy of the data and the practices of various stakeholders participating in the ecosystem.

### Example Data Flow of Components of a System

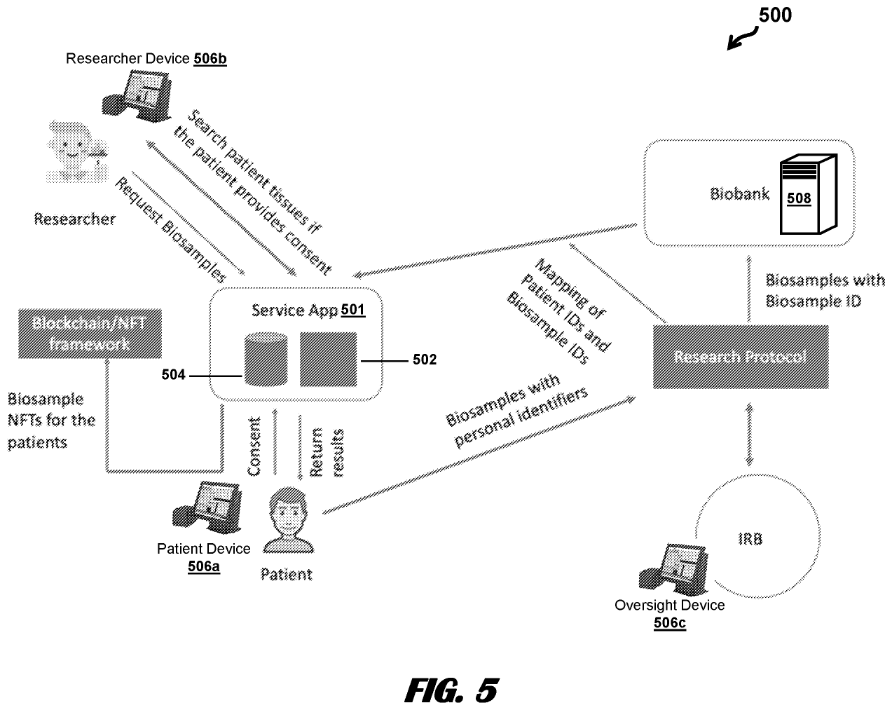

FIG. 5 depicts an example of data flow of components of a system 500 (such as the example system 100 of FIG. 1). The system 500 includes any number of client devices 506a-506c (collectively referred to as “client devices 506” or a “client device 506”) that function as nodes of a public or private blockchain, operated by end-users with various roles, such as patients, physicians or other healthcare providers, biomedical researchers, and oversight reviewers of Institutional Review Board (IRB) reviews, among others. The system 500 further includes a data service (e.g., authority system 101) that hosts a data service application (service app 501) and private centralized database 504, and manages the blockchain. The components of the system 500 access and implement the blockchain to track patient information. The system 500, for example, allows a biobank to upload various types of information about patients or biosamples to the service app 501 by accessing and employing the blockchain framework hosting NFTs.

Users or organizations register and enroll a user or enterprise account with the service app 501. A device of the system 500, such as the app server 502, client device 506, or other computing device, executes blockchain-related software routines to create a service account, blockchain account, and/or a new NFT representing the particular user or enterprise on the blockchain.

The client devices 506 include any computing device having computing hardware (e.g., processors, non-transitory machine-readable memory) and software for performing the various processes and tasks described herein. Non-limiting examples of the client devices 506 include servers, desktops, laptops, tablets, and the like. The client devices 506 include hardware and software configurations based upon the roles of the particular end-users. For example, the patient device 506a may access certain features of a cloud-app that are vary from the features accessed by a researcher device 506b (operated by a researcher or oversight device. As another example, oftentimes, an IRB approves research protocols that implement de-identification or other means of obfuscating personally identifying information. The oversight device 506c of an IRB user accesses features and functions of the cloud-app that permits the IRB users to approve or submit comments about research protocols, the types of information exchanged or available to researchers, among other oversight operations. As another example, biobankers can create a “biobanker account” via the website hosted by an app server 502, allowing a biobanker user to identify certain biosample information to provide to other users. Additionally or alternatively, the biobanker user (not shown) may instruct a biobank server 508 to upload selected biosample information, from a biobank database (not shown) to a service database 504.

In some implementations, the oversight device 506c or other device (e.g., research device, app server 502) may generate a research protocol sub-token for the researcher scaffold or patient scaffold indicating one or more types of data (e.g., attributes of samples, attributes of patients) expected or used for conducting the research. In some implementations, the oversight device 506c or other device (e.g., research device, app server 502) may generate an authentication protocol sub-token indicating authentication techniques and thresholds for permitting the researcher device (or other device) to access or distribute the digital or physical biospecimens and related biospecimen sub-tokens. For instance, a researcher token may be associated with sub-tokens representing the researcher's research protocols. The research protocols themselves will contain source material to inform the research process (e.g., details about what samples are needed and which patient data may be most useful/needed to accompany the samples). The research protocol sub-token may include this research profile data or pointers to this research profile data stored on a separate non-transitory storage.

A device of the system 500 (e.g., app server 502, client device 506) creates a new NFT for each biosample on the blockchain. The biosample NFT includes various types of information about the biosamaple, including a biosample identifier that uniquely identifies the biosample. The user account or, in some implementations the user's wallet, becomes the owner of the new biosample NFT. The blockchain entries capture the biosample identifiers of the biosamples and information about the users and/or entities that accessed, possessed, tested, or otherwise associated with the biosample. In the example system 500, certain types of sensitive information about the biosamples are not included in the public blockchain/NFT framework. Rather, such types of sensitive information are stored privately, off-chain, in the private database 504 of the service app 501 maintained by the host service. The private service database 504 of the service website contains the details of the biosamples, including information about which biosamples correspond to which patients.

Using the patient devices 506a, the patients can log in to the website or cloud-based service app 501 to access and review details of biosamples associated with the patient. The service app 501 allows the patients to track and review the studies or tests performed using the biosamples associated with the patient. The results of conducting research, tests, and other outcomes of procedures involving the patient's biosamples are returned to the patient or otherwise made accessible to the patient device 506a based on the information stored in the service database 504 through the service app 501. The patient may enter instructions to the service app 501 electing to make the patient's biosamples “searchable” by researchers involved with the system 500, thereby permitting the research devices 506b and/or the server 502 to query the service database 504 for the patient's biosamples and return biosample information to the researcher devices 506b. Alternatively, the patient may enter instructions to the server app 501 electing to make the patient's biosamples private (e.g., not searchable by researchers) from the researchers of the system 500, thereby disallowing the research devices 506b and/or the server 502 from querying the service database 504 for the patient's biosamples and returning the biosample information to the researcher devices 506b.

Using the researcher devices 506b, the researchers log in to the website or cloud-based service app 501 to access and search the service database 504 or the blockchain entries for the biosamples tested or analyzed by the researchers, or for potential biosamples that may contribute to proposed or ongoing research efforts. In some cases, the research device 506b uploads research results associated with a biosample to the private database 504. Additionally or alternatively, the research device 506b uploads or mints the research results associated with the biosample to a new results token containing information about the underlying biosample used in the research, such as a biospecimen identifier.

In some implementations, certain features require an informed consent indicator be inputted by the patient via the patient device 506a. The consent indicator permits access to patient or biosample information to other devices of the system 500 (e.g., researcher devices 506b, biobank server 508), unlocking functions for research use, consent to be able to be connected to track the outcomes of the use of the biosamples, and return of results to patients or physicians, thereby engaging patients as key stakeholders in how patient biosamples are used and referenced.

The service app 501 and components of the system 500 implement a public blockchain having the various types of NFTs (e.g., biosample NFTs, user NFTs, patient NFTs), where some or all o of the blockchain and the blockchain entries are publicly available, where the data in each blockchain entry remains encrypted or otherwise protected. The private centralized service database 504 and other aspects of the system 500 remain private and store data privately, off-chain, in non-transitory storage media. In some implementations, the service 501 and components of the system 500 implement a private blockchain having the various types of NFTs (e.g., biosample NFTs, user NFTs, patient NFTs), and the service database 504 and various components of the system 500 remain private and/or centralized, under the control of the data service. For instance, in some cases, the use of public blockchains is a privacy concern, or the system 500 needs to be tested in a very controlled setting. In such cases, the use of private blockchains and/or the private centralized service database 504 could be an alternate approach to achieve the objectives of the system 500.

In some embodiments, a computer-implemented for managing data for a blockchain, the method comprising: receiving, by a computer from a client device, patient personal data for a patient and a set of one or more validating inputs having validation data associated with one or more corresponding validating users; responsive to the computer validating the validation data of a threshold number of validation inputs from the set of one or more validation inputs, generating, by the computer, a patient token as a non-fungible token on a blockchain, the patient token representing the patient personal data and including a unique patient token identifier; receiving, by the computer from the client device, specimen data for a specimen associated with the patient, the specimen data including a unique specimen identifier based at least in part on the patient personal data; and generating, by the computer, a specimen sub-token of the blockchain representing the specimen data using at least a portion of the patient personal data of the patient token, the specimen sub-token including the unique specimen identifier, a unique specimen token identifier, the unique patient token identifier, and an encrypted version of the portion of the patient personal data.

In some implementations, the patient personal data comprises a patient unique identifier.

In some implementations, the specimen sub-token is a second non-fungible token of the blockchain.

In some implementations, receiving the specimen data includes: receiving, by the computer, a second set of one or more validating inputs having the validation data associated with the one or more corresponding validating users, wherein the computer generates the specimen sub-token responsive to the computer validating the validation data of the threshold number of validation inputs from the second set of one or more validation inputs.

In some implementations, the method further comprising, for each validation input, identifying, by the computer, a valid cryptographic certificate corresponding to the validation data of the validation input according to a certificate authority.

In some implementations, the patient token or the specimen sub-token includes at least one of: text data, media data, and a pointer for a remote non-transitory storage.

In some implementations, the remote non-transitory storage includes a biobank database containing at least a portion of the specimen data.

In some implementations, a first portion of the specimen data represented by the specimen sub-token is stored in the sub-token and a second portion of the specimen data represented by the specimen sub-token is stored in a database hosted on a non-transitory machine-readable memory.

In some implementations, further comprising receiving, by the computer, from a second client device treatment data for one or more treatments associated with one or more physicians; and generating, by the computer, a treatment sub-token representing the treatment data for a particular treatment using at least one of the patient data and the specimen data, responsive to the computer validating the validation data of the threshold number of validation inputs from a second set of one or more validation inputs.

In some implementations, further comprising obtaining, by the computer, a specimen request associated with a researcher device, the specimen request including one or more specimen attributes; querying, by the computer, the specimen data of a plurality of specimen sub-tokens using the one or more specimen attributes of the specimen request; determining, by the computer, a cohort of one or more specimens having the specimen data satisfying the one or more specimen attributes; generating, by the computer, one or more custody updates to one or more transaction blocks of the blockchain having the patient token, the one or more custody updates corresponding to the one or more specimens of the cohort.

In some implementations, obtaining the research request includes receiving, by the computer, from a second client device research data associated with the specimen according to one or more identifiers in the research data.

In some implementations, the computer obtains the research request from a non-transitory storage media, and wherein the computer is configured to generate the cohort at a predetermined interval using the research request stored in the storage media.

In some implementations, further comprising transmitting, by the computer, to a patient device, a prompt for display at a user interface of the patient device for prompting the patient to claim the patient token or the specimen sub-token; receiving, by the computer, from the patient device a claim instruction and a patient validating input; and in response to the computer validating patient validation data of the patient validating input: updating, by the computer, one or more transaction tokens of the blockchain to indicate a scaffold association between the patient token or the specimen sub-token and one or more identifiers associated with one or more patients or one or more patient tokens.

In some implementations, further comprising updating, by the computer, sub-token inventory data associated with the patient token to include the specimen unique identifiers of the specimen sub-token.

In some implementations, the patient includes a designated representative associated with the patient according to the patient personal data.

In some embodiments, a system comprises a computer comprising a processor configured to: receive, from a client device, patient personal data for a patient and a set of one or more validating inputs having validation data associated with one or more corresponding validating users; responsive to validating the validation data of a threshold number of validation inputs from the set of one or more validation inputs, generate a patient token as a non-fungible token on a blockchain, the patient token representing the patient personal data and including a unique patient token identifier; receive, from the client device, specimen data for a specimen associated with the patient, the specimen data including a unique specimen identifier based at least in part on the patient personal data; and generate a specimen sub-token of the blockchain representing the specimen data using at least a portion of the patient personal data of the patient token, the specimen sub-token including the unique specimen identifier, a unique specimen token identifier, the unique patient token identifier, and an encrypted version of the portion of the patient personal data.

In some implementations, the computer is further configured to receive a second set of one or more validating inputs having the validation data associated with the one or more corresponding validating users, and wherein the computer generates the specimen sub-token responsive to the computer validating the validation data of the threshold number of validation inputs from the second set of one or more validation inputs.

In some implementations, for each validation input, the computer is further configured to identify a valid cryptographic certificate corresponding to the validation data of the validation input according to a certificate authority.

In some implementations, the patient token or the specimen sub-token includes at least one of: text data, media data, and a pointer for a remote non-transitory storage.

In some implementations, the remote non-transitory storage includes a biobank database containing at least a portion of the specimen data.

In some embodiments, a computer-implemented method for managing data access for blockchain data, and the method comprises obtaining, by the computer, a specimen request associated with a researcher device, the specimen request including one or more specimen attributes; determining, by the computer, a cohort of one or more specimens having specimen data satisfying the one or more specimen attributes by querying the specimen data of a plurality of specimen sub-tokens of one or more blockchains using the one or more specimen attributes of the specimen request; generating, by the computer, one or more custody updates to one or more transaction blocks on a blockchain having the patient token, the one or more custody updates corresponding to the one or more specimens of the cohort, wherein the researcher device does not have access to patient personal data and a unique patient identifier; receiving, by a computer, from the researcher device research results data for a specimen of the cohort, the research results data including a unique specimen identifier for the specimen; and generating, by the computer, a research results sub-token representing the research results data using at least a portion of the research results data and a specimen unique identifier associated with a patient unique identifier of a patient token.

In some implementations, obtaining the research request includes receiving, by the computer, from a second client device research data associated with the specimen according to one or more identifiers in the research data.

In some implementations, the computer queries a taxonomy data structure associated with an attribute of the researcher when querying the specimen data of the plurality of specimen sub-tokens using the one or more specimen attributes.

In some implementations, the computer executes a smart contract associated with the blockchain, the smart contract comprising machine-readable instructs instructing the computer to generate the one or more custody updates.

In some implementations, further comprising determining, by the computer, the patient token associated with the specimen sub-token or the research results sub-token using one or more unique identifiers and one or more cryptographic values; generating, by the computer, one or more new credit values for the patient token responsive to the computer receiving from the researcher device at least one of the research request and the research results data; and updating, by the computer, a patient credit account according to the one or more new credit values.

In some implementations, further comprising generating, by the computer, a specimen sub-token representing the specimen data for the specimen and associated with the patient token using the at least a portion of the research results data and the specimen unique identifier.

In some implementations, further comprising transmitting, by the computer, to a patient device, a prompt for display at a user interface of the patient device and prompting the patient to claim the patient token or the specimen sub-token; receiving, by the computer, from the patient device a claim instruction and a patient validating input; and in response to the computer validating patient validation data of the patient validating input: updating, by the computer, one or more transaction blocks of the blockchain to indicate a scaffold association between the patient token or the specimen sub-token and one or more identifiers associated with the patient

In some implementations, generating, by the computer, a researcher token for one or more blockchains representing researcher profile data; determining, by the computer, a set of suggested study attributes based upon comparing the specimen data in a plurality of specimen sub-tokens, the patient data of a plurality patient tokens, and the researcher data of the researcher token; and identifying, by the computer, a suggested research cohort of one or more specimens associated with specimen data satisfying the set of suggested study attributes by querying the plurality of specimen sub-tokens according to the set of suggested study attributes.

In some implementations, the computer obtains a research protocol sub-token associated with the researcher device with the specimen request, wherein the research protocol sub-token includes a research profile indicating the specimen data and the one or more specimen attributes, and wherein the computer determines the cohort of one or more specimens using the research protocol sub-token.

In some implementations, the computer obtains a consent sub-token including identifying information for the patient associated with the specimen and a consent indicator.

In some embodiments, a system comprises a computer configured to obtain a specimen request associated with a researcher device, the specimen request including one or more specimen attributes; determine a cohort of one or more specimens having specimen data satisfying the one or more specimen attributes by querying the specimen data of a plurality of specimen sub-tokens of one or more blockchains using the one or more specimen attributes of the specimen request; generate one or more custody updates to one or more transaction blocks on a blockchain having the patient token, the one or more custody updates corresponding to the one or more specimens of the cohort, wherein the researcher device does not have access to patient personal data and a unique patient identifier; receive from the researcher device research results data for a specimen of the cohort, the research results data including a unique specimen identifier for the specimen; and generate a research results sub-token representing the research results data using at least a portion of the research results data and a specimen unique identifier associated with a patient unique identifier of a patient token.

In some implementations, when obtaining the research request the computer is configured to receive from a second client device research data associated with the specimen according to one or more identifiers in the research data.

In some implementations, the computer queries a taxonomy data structure associated with an attribute of the researcher when querying the specimen data of the plurality of specimen sub-tokens using the one or more specimen attributes.

In some implementations, the computer executes a smart contract associated with the blockchain, the smart contract comprising machine-readable instructs instructing the computer to generate the one or more custody or access privilege updates.

In some implementations, further comprising determining, by the computer, the patient token associated with the specimen sub-token or the research results sub-token using one or more unique identifiers and one or more cryptographic values; and generating, by the computer, one or more new credit values for the patient token responsive to the computer receiving from the researcher device at least one of the research request and the research results data; and updating, by the computer, a patient credit account according to the one or more new credit values.

In some implementations, further comprising generating, by the computer, a specimen sub-token representing the specimen data for the specimen and associated with the patient token using the at least a portion of the research results data and the specimen unique identifier.

In some implementations, further comprising transmitting, by the computer, to a patient device, a prompt for display at a user interface of the patient device and prompting the patient to claim the patient token or the specimen sub-token; and receiving, by the computer, from the patient device a claim instruction and a patient validating input; and in response to the computer validating patient validation data of the patient validating input: updating, by the computer, one or more transaction blocks of the blockchain to indicate a scaffold association between the patient token or the specimen sub-token and one or more identifiers associated with the patient.

In some implementations, further comprising generating, by the computer, a researcher token for one or more blockchains representing researcher profile data; and determining, by the computer, a set of suggested study attributes based upon comparing the specimen data in a plurality of specimen sub-tokens, the patient data of a plurality patient tokens, and the researcher data of the researcher token; and identifying, by the computer, a suggested research cohort of one or more specimens associated with specimen data satisfying the set of suggested study attributes by querying the plurality of specimen sub-tokens according to the set of suggested study attributes.

In some implementations, the computer obtains a research protocol sub-token associated with the researcher device with the specimen request, wherein the research protocol sub-token includes a research profile indicating the specimen data and the one or more specimen attributes, and wherein the computer determines the cohort of one or more specimens using the research protocol sub-token.

In some implementations, the computer obtains a consent sub-token including identifying information for the patient associated with the specimen and a consent indicator.

In some embodiments, a computer-implemented method comprises receiving, by a computer, research data or treatment data for a patient represented by a patient token of a blockchain, a physician identifier, and a set of one or more validating inputs purportedly validating the research data or treatment data from a physician device; generating, by the computer, a treatment sub-token representing the research data or treatment data using a physician unique identifier and at least a portion of patient data for the patient, responsive to the computer validating the validation data of a threshold number of validation inputs from the set of one or more validation inputs; receiving, by the computer, from a patient device a claim instruction and a patient validating input; and in response to the computer validating patient validation data of the patient validating input: updating, by the computer, one or more transaction blocks of the blockchain to indicate an association between the patient token or the treatment sub-token and one or more identifiers associated with the patient.

In some implementations, receiving the claim instruction includes: transmitting, by the computer, to the patient device, a prompt for display at a user interface of the patient device for prompting the patient to claim the treatment sub-token, wherein the computer receives the claim instruction via the user interface of the patient device.

In some implementations, the treatment sub-token includes relationship data representing a therapeutic relationship with a physician, the relationship data of the treatment sub-token including a unique physician identifier.

In some implementations, further comprising receiving, by a computer, from the physician device specimen data for a specimen associated with the patient, the specimen data including a unique specimen identifier based at least in part on a unique patient identifier; generating, by the computer, a specimen sub-token representing the specimen data using at least a portion of the patient data of the patient token, the specimen sub-token including the unique specimen identifier and an encrypted version of the unique patient identifier.

In some implementations, receiving the specimen data includes: receiving, by the computer, a second set of one or more validating inputs having the validation data associated with the one or more corresponding validating users, wherein the computer generates the specimen sub-token responsive to the computer validating the validation data of the threshold number of validation inputs from the second set of one or more validation inputs.

In some implementations, receiving the treatment data includes: receiving, by a computer, from the physician device a physician access request for a patient token and a plurality of sub-tokens of a patient scaffold; and receiving, by the computer, a validating input for the physician for validating the physician access request, wherein the computer receives the treatment data based upon successfully authenticating the physician according to the validating input for the physician.

In some implementations, receiving the treatment data includes: receiving, by a computer, from the physician device a physician access request for a patient token and a plurality of sub-tokens of a patient scaffold; and receiving, by the computer, a validating input for the physician for validating the physician access request, wherein the computer receives the treatment data based upon successfully authenticating the physician according to the physician token.

In some implementations, further comprising: generating, by the computer, a physician token for one or more blockchains representing physician profile data; and determining, by the computer, a set of suggested physician attributes based upon comparing the physician data in a plurality of physician tokens and the patient data of the patient token; and identifying, by the computer, a suggested set of one or more physicians associated with the physician profile data satisfying the set of suggested physician attributes by querying the plurality of physician tokens according to the set of suggested physician attributes.

In some implementations, further comprising: receiving, by the computer, research results data from a researcher device for a specimen associated with the patient; and transmitting, by the computer, the research results data to the physician device for display via a user interface of the physician device.

In some implementations, the research results data includes a graph of one or more sub-tokens containing the research results data for one or more specimens.

In some embodiments, a system comprises a computer comprising a processor configured to: receive research data or treatment data for a patient represented by a patient token of a blockchain, a physician identifier, and a set of one or more validating inputs purportedly validating the research data or treatment data from a physician device; generate a treatment sub-token representing the research data or treatment data using a physician unique identifier and at least a portion of patient data for the patient, responsive to the computer validating the validation data of a threshold number of validation inputs from the set of one or more validation inputs; receive from a patient device a claim instruction and a patient validating input; and in response to the computer validating patient validation data of the patient validating input: update one or more transaction blocks of the blockchain to indicate an association between the patient token or the treatment sub-token and one or more identifiers associated with the patient.

In some implementations, the computer is further configured to transmit, to the patient device, a prompt for display at a user interface of the patient device for prompting the patient to claim the treatment sub-token, wherein the computer receives the claim instruction via the user interface of the patient device.

In some implementations, the treatment sub-token includes relationship data representing a therapeutic relationship with a physician, the relationship data of the treatment sub-token including a unique physician identifier.

In some implementations, the computer is further configured to: receive, from the physician device specimen data for a specimen associated with the patient, the specimen data including a unique specimen identifier based at least in part on a unique patient identifier; and generate a specimen sub-token representing the specimen data using at least a portion of the patient data of the patient token, the specimen sub-token including the unique specimen identifier and an encrypted version of the unique patient identifier

In some implementations, when receiving the specimen data the computer is further configured to receive a second set of one or more validating inputs having the validation data associated with the one or more corresponding validating users, and wherein the computer generates the specimen sub-token responsive to the computer validating the validation data of the threshold number of validation inputs from the second set of one or more validation inputs.

In some implementations, when receiving the treatment data the computer is further configured to: receive from the physician device a physician access request for a patient token and a plurality of sub-tokens of a patient scaffold; and receive a validating input for the physician for validating the physician access request, wherein the computer receives the treatment data based upon successfully authenticating the physician according to the validating input for the physician.

In some implementations, when receiving the treatment data the computer is further configured to receive from the physician device a physician access request for a patient token and a plurality of sub-tokens of a patient scaffold; and receive a validating input for the physician for validating the physician access request, wherein the computer receives the treatment data based upon successfully authenticating the physician according to a physician token.

In some implementations, the computer is further configured to generate a physician token for one or more blockchains representing physician profile data; determine a set of suggested physician attributes based upon comparing the physician data in a plurality of physician tokens and the patient data of the patient token; and identify a suggested set of one or more physicians associated with the physician profile data satisfying the set of suggested physician attributes by querying the plurality of physician tokens according to the set of suggested physician attributes.

In some implementations, the computer is further configured to: receive research results data from a researcher device for a specimen associated with the patient; and transmit the research results data to the physician device for display via a user interface of the physician device.

In some implementations, the research results data includes a graph of one or more sub-tokens containing the research results data for one or more specimens.

In some embodiments, a computer-implemented for managing data for a blockchain, where the method comprising: receiving, by a computer from a client device, physician data for a healthcare provider and a set of one or more validating inputs having validation data associated with one or more corresponding validating users; responsive to the computer validating the validation data of a threshold number of validation inputs from the set of one or more validation inputs, generating, by the computer, a physician token as a non-fungible token on a blockchain, the physician token representing the physician and including physician profile information of the physician data, a unique physician identifier, and a unique physician token identifier; and generating, by the computer, one or more physician-related sub-tokens of the blockchain representing at least a portion of the physician data, each physician-related sub-token including a unique sub-token identifier, the unique physician token identifier, and an encrypted version of the unique physician identifier.

In some implementations, the physician-related data includes location data indicating a location of practice of the physician, wherein the one or more physician sub-tokens includes a healthcare site sub-token representing the location of practice of the physician, and wherein the healthcare site sub-token includes the location data in the physician data.

In some implementations, the one or more physician-related sub-tokens includes a treatment sub-token representing a therapeutic relationship with a patient, the treatment token including a unique patient identifier, and the method further comprising: receiving, by the computer from the client device, patient data and treatment data associated with the patient, including the unique patient identifier and treatment information; and generating, by the computer, the treatment sub-token representing the treatment data using the physician unique identifier and at least a portion of the patient data for the patient.

In some implementations, the computer generates the treatment sub-token responsive to the computer validating second validation data of the threshold number of the validation inputs from a second set of one or more validation inputs.

In some embodiments, a system comprises a computer comprising a processor configured to: receive from a client device physician data for a healthcare provider and a set of one or more validating inputs having validation data associated with one or more corresponding validating users; responsive to the computer validating the validation data of a threshold number of validation inputs from the set of one or more validation inputs, generate a physician token as a non-fungible token on a blockchain, the physician token representing the physician and including physician profile information of the physician data, a unique physician identifier, and a unique physician token identifier; and generate one or more physician-related sub-tokens of the blockchain representing at least a portion of the physician data, each physician sub-token including a unique sub-token identifier, the unique physician token identifier, and an encrypted version of the unique physician identifier.

In some implementations, the physician data includes location data indicating a location of practice of the physician, wherein the one or more physician-related sub-tokens includes a healthcare site sub-token representing the location of practice of the physician, and wherein the healthcare site sub-token includes the location data in the physician data.

In some implementations, the one or more physician-related sub-tokens includes a treatment sub-token representing a therapeutic relationship with a patient, the treatment token including a unique patient identifier, and wherein the computer is further configured to: receive from the client device patient data and treatment data associated with the patient, including the unique patient identifier and treatment information; and generate the treatment sub-token representing the treatment data using the physician unique identifier and at least a portion of the patient data for the patient.

In some implementations, the computer generates the treatment sub-token responsive to the computer validating second validation data of the threshold number of the validation inputs from a second set of one or more validation inputs.

In some embodiments, a computer-implemented for managing data for a blockchain, and the method comprises receiving, by a computer from a client device, researcher data for a researcher and a set of one or more validating inputs having validation data associated with one or more corresponding validating users; responsive to the computer validating the validation data of a threshold number of validation inputs from the set of one or more validation inputs, generating, by the computer, a researcher token as a non-fungible token on a blockchain, the researcher token representing the researcher and including researcher profile information of the researcher data, a unique researcher identifier, and a unique researcher token identifier; generating, by the computer, one or more researcher sub-tokens of the blockchain representing at least a portion of the researcher data, each researcher sub-token including a unique sub-token identifier, the unique researcher token identifier, and an encrypted version of the unique researcher identifier.

In some implementations, the researcher data includes laboratory data containing laboratory information for a laboratory of the researcher, wherein the one or more researcher sub-tokens includes a laboratory sub-token representing the laboratory of the researcher, and wherein the researcher sub-token includes the laboratory data of the researcher data.

In some implementations, the one or more researcher sub-tokens includes an authentication protocol sub-token representing an authentication protocol executed by the computer for permitting the researcher to collect one or more specimens for one or more patients, the protocol sub-token including one or more oversight user identifiers and indicating an authentication technique for the authentication protocol.

In some implementations, the one or more researcher sub-tokens includes a research protocol sub-token representing a research protocol, including research information indicating one or more specimen attributes and one or more types of patient data used for the research protocol.

In some embodiments, a system comprises a computer comprising a processor and configured to: receive from a client device researcher data for a researcher and a set of one or more validating inputs having validation data associated with one or more corresponding validating users; responsive to the computer validating the validation data of a threshold number of validation inputs from the set of one or more validation inputs, generate a researcher token as a non-fungible token on a blockchain, the researcher token representing the researcher and including researcher profile information of the researcher data, a unique researcher identifier, and a unique researcher token identifier; and generate one or more researcher sub-tokens of the blockchain representing at least a portion of the researcher data, each researcher sub-token including a unique sub-token identifier, the unique researcher token identifier, and an encrypted version of the unique researcher identifier.

In some implementations, the researcher data includes laboratory data containing laboratory information for a laboratory of the researcher, wherein the one or more researcher sub-tokens includes a laboratory sub-token representing the laboratory of the researcher, and wherein the researcher sub-token includes the laboratory data of the researcher data.

In some implementations, the one or more researcher sub-tokens includes an authentication protocol sub-token representing an authentication protocol executed by the computer for permitting the researcher to collect one or more specimens for one or more patients, the protocol sub-token including one or more oversight user identifiers and indicating an authentication technique for the authentication protocol.

In some implementations, wherein the one or more researcher sub-tokens includes a research protocol sub-token representing a research protocol, including research information indicating one or more specimen attributes and one or more types of patient data used for the research protocol.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, attributes, or memory contents. Information, arguments, attributes, data, etc. may be passed, forwarded, or transmitted via any suitable means including memory sharing, message passing, token passing, network transmission, etc.

The actual software code or specialized control hardware used to implement these systems and methods is not limiting of the invention. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

1.-76. (canceled)

77. A computer-implemented method for managing data for a blockchain, the method comprising:
receiving, by a computer from a client device, patient personal data for a patient and a set of one or more validating inputs having validation data associated with one or more corresponding validating users;
responsive to the computer validating the validation data of a threshold number of validation inputs from the set of one or more validation inputs, generating, by the computer, a patient token as a non-fungible token on a blockchain, the patient token representing the patient personal data and including a unique patient token identifier;
receiving, by the computer from the client device, specimen data for a specimen associated with the patient, the specimen data including a unique specimen identifier based at least in part on the patient personal data; and
generating, by the computer, a specimen sub-token of the blockchain representing the specimen data using at least a portion of the patient personal data of the patient token, the specimen sub-token including the unique specimen identifier, a unique specimen token identifier, the unique patient token identifier, and an encrypted version of the portion of the patient personal data.

78. The method according to claim 77, wherein the patient personal data comprises a patient unique identifier.

79. The method according to claim 77, wherein the specimen sub-token is a second non-fungible token of the blockchain.

80. The method according to claim 77, wherein receiving the specimen data includes:
receiving, by the computer, a second set of one or more validating inputs having the validation data associated with the one or more corresponding validating users,
wherein the computer generates the specimen sub-token responsive to the computer validating the validation data of the threshold number of validation inputs from the second set of one or more validation inputs.

81. The method according to claim 77, further comprising, for each validation input, identifying, by the computer, a valid cryptographic certificate corresponding to the validation data of the validation input according to a certificate authority.

82. The method according to claim 77, wherein the patient token or the specimen sub-token includes at least one of: text data, media data, and a pointer for a remote non-transitory storage.

83. The method according to claim 82, wherein the remote non-transitory storage includes a biobank database containing at least a portion of the specimen data.

84. The method according to claim 77, wherein a first portion of the specimen data represented by the specimen sub-token is stored in the sub-token and a second portion of the specimen data represented by the specimen sub-token is stored in a database hosted on a non-transitory machine-readable memory.

85. The method according to claim 77, further comprising:
receiving, by the computer, from a second client device treatment data for one or more treatments associated with one or more physicians; and
generating, by the computer, a treatment sub-token representing the treatment data for a particular treatment using at least one of the patient data and the specimen data, responsive to the computer validating the validation data of the threshold number of validation inputs from a second set of one or more validation inputs.

86. The method according to claim 77, further comprising:
obtaining, by the computer, a specimen request associated with a researcher device, the specimen request including one or more specimen attributes;
querying, by the computer, the specimen data of a plurality of specimen sub-tokens using the one or more specimen attributes of the specimen request;
determining, by the computer, a cohort of one or more specimens having the specimen data satisfying the one or more specimen attributes;
generating, by the computer, one or more custody updates to one or more transaction blocks of the blockchain having the patient token, the one or more custody updates corresponding to the one or more specimens of the cohort.

87. The method according to claim 84, wherein obtaining the research request includes receiving, by the computer, from a second client device research data associated with the specimen according to one or more identifiers in the research data.

88. The method according to claim 84, wherein the computer obtains the research request from a non-transitory storage media, and wherein the computer is configured to generate the cohort at a predetermined interval using the research request stored in the storage media.

89. The method according to claim 77, further comprising:
transmitting, by the computer, to a patient device, a prompt for display at a user interface of the patient device for prompting the patient to claim the patient token or the specimen sub-token;
receiving, by the computer, from the patient device a claim instruction and a patient validating input; and
in response to the computer validating patient validation data of the patient validating input:
updating, by the computer, one or more transaction tokens of the blockchain to indicate a scaffold association between the patient token or the specimen sub-token and one or more identifiers associated with one or more patients or one or more patient tokens.

90. The method according to claim 77, further comprising updating, by the computer, sub-token inventory data associated with the patient token to include the specimen unique identifiers of the specimen sub-token.

91. The method according to claim 77, wherein the patient includes a designated representative associated with the patient according to the patient personal data.

92. A system comprising:
a computer comprising a processor configured to:
receive, from a client device, patient personal data for a patient and a set of one or more validating inputs having validation data associated with one or more corresponding validating users;
responsive to validating the validation data of a threshold number of validation inputs from the set of one or more validation inputs, generate a patient token as a non-fungible token on a blockchain, the patient token representing the patient personal data and including a unique patient token identifier;
receive, from the client device, specimen data for a specimen associated with the patient, the specimen data including a unique specimen identifier based at least in part on the patient personal data; and
generate a specimen sub-token of the blockchain representing the specimen data using at least a portion of the patient personal data of the patient token, the specimen sub-token including the unique specimen identifier, a unique specimen token identifier, the unique patient token identifier, and an encrypted version of the portion of the patient personal data.

93. The system according to claim 92, wherein the computer is further configured to receive a second set of one or more validating inputs having the validation data associated with the one or more corresponding validating users, and wherein the computer generates the specimen sub-token responsive to the computer validating the validation data of the threshold number of validation inputs from the second set of one or more validation inputs.

94. The system according to claim 92, wherein, for each validation input, the computer is further configured to identify a valid cryptographic certificate corresponding to the validation data of the validation input according to a certificate authority.

95. The system according to claim 92, wherein the patient token or the specimen sub-token includes at least one of: text data, media data, and a pointer for a remote non-transitory storage.

96. The method according to claim 92, wherein the remote non-transitory storage includes a biobank database containing at least a portion of the specimen data.
